(19) United States
(12) Patent Application Publication
(10) Pub. No.: US 2026/0078233 A1
(43) Pub. Date: Mar. 19, 2026

(54) ENZYMES, MICRO-ORGANISMS AND USES THEREOF, AND A METHOD OF DEGRADING HYDROCARBON CHAINS

(71) Applicant: Teknologian tutkimuskeskus VTT Oy, Espoo (FI)

(72) Inventors: Kari KOIVURANTA, VTT (FI); Gustavo BORIN, VTT (FI); Sandra CASTILLO, VTT (FI)

(21) Appl. No.: 19/102,689

(22) PCT Filed: Aug. 10, 2023
(86) PCT No.: PCT/FI2023/050461

§ 371 Date: Feb. 10, 2025

(30) Foreign Application Priority Data
Aug. 11, 2022 (FI) 20225715

(51) Int. Cl. C08J 11/10; C12N 1/14; C12N 1/20; C12N 9/48; C12N 15/52; C12N 15/63; C12R 1/01; C12R 1/645

(52) U.S. Cl. CPC C08J 11/105; C12N 1/14; C12N 1/20; C12N 9/485; C12N 15/52; C12N 15/63; C12Y 304/11024; C08J 2323/02; C08J 2325/04; C12R 2001/01; C12R 2001/645

(57) ABSTRACT

The present invention relates to the fields of life sciences, micro-organisms and degradation of polyolefins and/or polystyrenes. Specifically, the invention relates to an isolated specific enzyme or a fragment thereof, wherein said enzyme or fragment is capable of degrading a polyolefin and/or a polystyrene, and to a micro-organism or a host cell comprising the enzyme or a fragment thereof. Also, the present invention relates to a polynucleotide encoding the enzyme or fragment thereof, and to an expression vector or plasmid comprising the polynucleotide of the present invention. And still, the present invention relates to use of the enzyme, fragment, micro-organ-ism, host cell, polynucleotide, expression vector or plasmid of the present invention for degrading a polyolefin and/or a polystyrene; to a method of degrading a polyolefin and/or a polystyrene with the specific enzyme or a fragment thereof; and to a method of producing the enzyme or fragment thereof of the present invention.

### FIELD OF THE INVENTION

The present invention relates to the fields of life sciences, micro-organisms and degradation of hydrocarbon chains such as polyolefins and polystyrene. Specifically, the invention relates to an isolated specific enzyme, or a fragment thereof, wherein said enzyme or fragment is capable of degrading a hydrocarbon chain such as a polyolefin and/or a polystyrene, and to a micro-organism or a host cell comprising the enzyme or a fragment thereof. Also, the present invention relates to a polynucleotide encoding the enzyme or fragment thereof, and to an expression vector or plasmid comprising the polynucleotide of the present invention. And still, the present invention relates to use of the enzyme, fragment, micro-organism, host cell, polynucleotide, expression vector or plasmid of the present invention for degrading a hydrocarbon chain such as a polyolefin and/or a polystyrene; to a method of degrading a hydrocarbon chain such as a polyolefin and/or a polystyrene with the specific enzyme or a fragment thereof; and to a method of producing the enzyme or fragment thereof of the present invention.

### BACKGROUND OF THE INVENTION

With the existing plastic recycling systems (mechanical and chemical) not all plastic waste can be recycled. This is partly due to the quality of plastic wastes (mixed plastic, dirty plastics). Additionally, the existing recycling methods need much energy. Biotechnical recycling could be utilized for improving the range of recycling methods and for enabling cost effective and more efficient recycling of plastics.

Removal of highly stable and durable hydrocarbon chains such as polyolefin and/or polystyrene polymers including but not limited to plastics comprising polyolefins and/or polystyrenes from the environment by using microbes or microbial enzymes is of high interest. In general, biotechnical plastic degradation is not common yet. Only few micro-organisms or enzymes capable of degrading polyolefins and/or polystyrenes have been discovered and said micro-organisms or enzymes are not effective. For example, Santo M. et al. (2013, International Biodeterioration & Biodegradation 84, 204-210) describe degradation of polyethylene (PE) with an extracellular fraction comprising different enzymes obtained from a Rhodococcus ruber cell culture. However, for PE or other polyolefins and/or polystyrene, recycling systems utilizing specific enzymes including but not limited to isolated and/or purified enzymes are under development. Indeed, it is very difficult to degrade hydrocarbon chains with enzymes.

Micro-organisms and enzymes are needed for rapid degradation and recycling of hydrocarbon chains. There remains a significant unmet need for specific micro-organisms and enzymes for effective degradation of hydrocarbon chains such as polyolefin and/or polystyrene polymers or plastics.

### BRIEF DESCRIPTION OF THE INVENTION

By biotechnical degradation and tools of the present invention it is possible to degrade and therefore recycle hydrocarbon chains such as plastics or synthetic polymers and more specifically polyolefins and/or polystyrenes. Furthermore, the tools of the present invention can be used e.g., for upcycling hydrocarbon chains i.e., for modifying a non-biodegradable plastic or polyolefin (e.g., PE) and/or polystyrene to a biodegradable plastic or fatty acid derived products by micro-organisms and enzymes.

The objects of the invention, namely methods and tools for degrading hydrocarbon chains such as polyolefins and/or polystyrenes are achieved by utilizing a specific enzyme or enzymes, or a specific micro-organism or micro-organisms (e.g. a bacterium/bacteria and/or fungus/fungi) comprising said enzyme(s).

The present invention provides methods and tools which enable biotechnical degradation of hydrocarbon chains such as polyolefins and/or polystyrenes. Said methods and tools provide surprising degradation effects on hydrocarbon chains such as polyolefins and/or polystyrenes or on a combination of specific plastics or polymers comprising polyolefins and/or polystyrenes. Also, the present invention can overcome the problems of the prior art including but not limited to ineffective or slow biotechnical degradation of polyolefin and/or polystyrene polymers. Furthermore, the specific enzyme or micro-organism of the present invention enable degradation methods at low temperatures, e.g., at a temperature below 100° C., indicating low energy need and costs.

Also, the inventors of the present disclosure surprisingly found out that unique or specific products can be obtained with the present invention. Further, the inventors noticed that unique or specific products can be produced from the degradation products of hydrocarbons, such as polyolefins and/or polystyrenes, by the enzymes, micro-organisms and/or host cells of the present invention.

Specifically, the present invention relates to a method of degrading a polyolefin and/or a polystyrene, said method comprising providing a material comprising a polyolefin and/or a polystyrene and an enzyme or a fragment thereof capable of degrading the polyolefin and/or the polystyrene, and allowing said enzyme or fragment thereof to degrade the polyolefin and/or the polystyrene, wherein the enzyme or the fragment thereof belongs to M28F peptidase subfamily and/or the enzyme or the fragment thereof has at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49.

In one embodiment, the present invention relates to a method of degrading a polyolefin and/or a polystyrene, said method comprising providing a material comprising a polyolefin and/or a polystyrene and an enzyme or a fragment thereof capable of degrading the polyolefin and/or the polystyrene, and allowing said enzyme or fragment thereof to degrade the polyolefin and/or the polystyrene, wherein the enzyme or fragment thereof belongs to M28F peptidase subfamily and comprises one or more amino acids selected from the group comprising Gly264, Ala265, Ser270, Gly271 and Gly305 corresponding to the amino acid positions presented in SEQ ID NO: 4, and/or the enzyme or fragment thereof has at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49.

In one embodiment, the present invention relates to a method of degrading a polyolefin and/or polystyrene, said method comprising
providing a material comprising a polyolefin and/or a polystyrene and an enzyme or a fragment thereof capable of degrading the polyolefin and/or the polystyrene, and
allowing said enzyme or fragment thereof to degrade the polyolefin and/or the polystyrene, wherein the enzyme or the fragment thereof belongs to M28F peptidase subfamily and comprises the amino acids Gly264, Ala265, Ser270, Gly271 and Gly305 corresponding to the amino acid positions presented in SEQ ID NO: 4, and/or the enzyme or fragment thereof has at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49.

In one embodiment, the enzyme or fragment thereof degrading a polyolefin and/or a polystyrene belongs to M28F peptidase subfamily and/or the enzyme or fragment thereof has at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49.

In one embodiment, the enzyme or the fragment thereof degrading a polyolefin and/or a polystyrene belongs to M28F peptidase subfamily and comprises one or more of the amino acids selected from the group Gly264, Ala265, Ser270, Gly271 and Gly305 corresponding to the amino acid positions presented in SEQ ID NO: 4 and/or the enzyme or fragment thereof has at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49.

In one embodiment, the enzyme or fragment thereof degrading a polyolefin and/or polystyrene belongs to M28F peptidase subfamily and comprises the amino acids Gly264, Ala265, Ser270, Gly271 and Gly305 corresponding to the amino acid positions presented in SEQ ID NO: 4 and/or the enzyme or fragment thereof has at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49.

Also, the present invention relates to an isolated enzyme or a fragment thereof belonging to M28F peptidase subfamily and/or the enzyme or fragment thereof has at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, wherein said enzyme or fragment is capable of degrading a polyolefin and/or a polystyrene.

In one embodiment, the present invention relates to an isolated enzyme or a fragment thereof belonging to M28F peptidase subfamily and comprising one or more amino acids selected from the group comprising Gly264, Ala265, Ser270, Gly271 and Gly305 corresponding to the amino acid positions presented in SEQ ID NO: 4, and/or the enzyme or the fragment thereof has at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, wherein said enzyme or fragment is capable of degrading a polyolefin and/or a polystyrene.

In one embodiment, the present invention relates to an isolated enzyme or a fragment thereof belonging to M28F peptidase subfamily and comprising the amino acids Gly264, Ala265, Ser270, Gly271 and Gly305 corresponding to the amino acid positions presented in SEQ ID NO: 4, and/or the enzyme or fragment thereof has at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, wherein said enzyme or fragment is capable of degrading a polyolefin and/or a polystyrene.

Furthermore, the present invention relates to a micro-organism or a host cell comprising an enzyme or a fragment thereof belonging to M28F peptidase subfamily and/or having at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, wherein said enzyme or fragment is capable of degrading a polyolefin and/or a polystyrene.

In one embodiment, the present invention relates to a micro-organism or a host cell comprising an isolated enzyme or a fragment thereof belonging to M28F peptidase subfamily and comprising one or more amino acids selected from the group comprising Gly264, Ala265, Ser270, Gly271 and Gly305 corresponding to the amino acid positions presented in SEQ ID NO: 4, and/or the enzyme or fragment thereof has at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, wherein said enzyme or fragment is capable of degrading a polyolefin and/or a polystyrene.

In one embodiment, the present invention relates to a micro-organism or a host cell comprising an isolated enzyme or a fragment thereof belonging to M28F peptidase subfamily and comprising the amino acids Gly264, Ala265, Ser270, Gly271 and Gly305 corresponding to the amino acid positions presented in SEQ ID NO: 4, and/or the enzyme or fragment thereof has at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, wherein said enzyme or fragment is capable of degrading a polyolefin and/or polystyrene.

Still, the present invention relates to a polynucleotide encoding the enzyme or fragment thereof of the present invention.

Still, the present invention relates to an expression vector or plasmid comprising the polynucleotide of the present invention.

And still, the present invention relates to use of the enzyme, fragment, micro-organism, host cell, polynucleotide, expression vector or plasmid of the present invention or any combination thereof for degrading a polyolefin and/or a polystyrene.

Still furthermore, the present invention relates to a method of producing the enzyme or fragment thereof of the present invention, wherein a recombinant micro-organism or host cell comprising the polynucleotide encoding the enzyme or fragment thereof of the present invention is allowed to express said enzyme or fragment thereof.

Other objects, details and advantages of the present invention will become apparent from the following drawings, detailed description and examples.

The objects of the invention are achieved by enzymes, micro-organisms, uses and methods characterized by what is stated in the independent claims. The preferred embodiments of the invention are disclosed in the dependent claims.

### BRIEF DESCRIPTION OF THE DRAWINGS

FIG. 1 shows results from the GC-MS run. With Bacillus licheniformis enzyme sample several peaks appeared which were missing from control sample (in controls an empty plasmid) with polypropylene (PP) powder.

FIG. 2 shows results from the GC-MS run. With Bacillus licheniformis enzyme sample several peaks appeared which were missing from control samples (in control an empty plasmid) with polyethylene (PE) powder.

FIG. 3 shows results from the GC-MS run. With Bacillus licheniformis enzyme sample several peaks including styrene peak, were larger than in control sample (in control an empty plasmid) with polystyrene (PS) powder.

FIG. 4 shows functional domains predicted by Pfam in Bacillus licheniformis (SEQ ID NO: 4), Bacillus flexus (SEQ ID NO:2), Bacillus subtilis (SEQ ID NO:6) and Bacillus cereus (SEQ ID NO:8) M28 family peptidases. PA corresponds to protease-associated (PF02225) domain and peptidase_M28 peptidase to family M28 (PF04389) domain.

FIG. 5 shows an alignment of several amino acid sequences of micro-organisms and consensus amino acids based on Bacillus licheniformis M28 family peptidase amino acid positions Gly264, Ala265, Ser270, Gly271 and Gly305.

FIG. 6 shows a pairwise alignment of Bacillus licheniformis M28 family peptidase (SEQ ID NO: 4) and B. thuringiensis M28 family peptidase (SEQ ID NO: 39). Detected consensus amino acids have been marked with bold in both amino acid sequences.

FIG. 7 shows two-dimensional structure (alfa helixes and beta sheets) of Bacillus licheniformis M28 family peptidase (SEQ ID NO: 4) and localisation of consensus amino acids. Alfa helixes are underlined and numbered with Arabic numbers. Beta sheets are in Italics and numbered with Roman numbers. Consensus amino acids are in bold. Amino acids presenting protease associated (PA) domain (amino acids 101-225) and which are removed from truncated Bacillus licheniformis M28 family peptidase are marked on grey.

FIG. 8 shows results from the FTIR analysis. With Bacillus licheniformis truncated enzyme sample several peaks appeared which were missing from control sample (in controls denaturated enzyme sample) with polystyrene (PS) powder.

FIG. 9 shows a plasmid map of pPB114-1.

### SEQUENCE LISTING

SEQ ID NO: 1: Bacillus flexus M28 family peptidase nucleotide sequence;
SEQ ID NO: 2: Bacillus flexus M28 family peptidase amino acid sequence;
SEQ ID NO: 3: Bacillus licheniformis M28 family peptidase nucleotide sequence;
SEQ ID NO: 4: Bacillus licheniformis M28 family peptidase amino acid sequence;
SEQ ID NO: 5: Bacillus subtilis M28 family peptidase nucleotide sequence;
SEQ ID NO: 6: Bacillus subtilis M28 family peptidase amino acid sequence;
SEQ ID NO: 7: Bacillus cereus M28 family peptidase nucleotide sequence;
SEQ ID NO: 8: Bacillus cereus M28 family peptidase amino acid sequence;
SEQ ID NO: 9: oPlastBug-134 oligonucleotide;
SEQ ID NO: 10: oPlastBug-135 oligonucleotide;
SEQ ID NO: 11: oPlastBug-124 oligonucleotide;
SEQ ID NO: 12: oPlastBug-125 oligonucleotide;
SEQ ID NO: 13: Bacillus amyloliquefaciens M28 family peptidase amino acid sequence ABS75456, MEROPS ID: MER0023566;
SEQ ID NO: 14: Bacillus cereus M28 family peptidase amino acid sequence EJS00819, MEROPS ID: MER0151534;
SEQ ID NO: 15: Bacillus sp. M28 family peptidase amino acid sequence ZP_08004478, MEROPS ID: MER0194201;
SEQ ID NO: 16: Bacillus sp. M28 family peptidase amino acid sequence ZP_10044385, MEROPS ID: MER0195433;
SEQ ID NO: 17: Bacillus sp. M28 family peptidase amino acid sequence ZP 10386958, MEROPS ID: MER0151611;
SEQ ID NO: 18: Bacillus subtilis M28 family peptidase amino acid sequence UniProt P25152, MEROPS: MER0001289;
SEQ ID NO: 19: Bacillus sp. M28 family peptidase amino acid sequence ZP_080084151, MEROPS ID: MER0194157;
SEQ ID NO: 20: Bacillus sp. M28 family peptidase amino acid sequence EAR65944, MEROPS ID: MER0079049;
SEQ ID NO: 21: Bacillus cereus M28 family peptidase amino acid sequence ZP 04219960, MEROPS ID: MER0190232;
SEQ ID NO: 22: Bacillus mycoides M28 family peptidase amino acid sequence ZP 04159713, MEROPS ID: MER0190222;
SEQ ID NO: 23: Bacillus sp. M28 family peptidase amino acid sequence ZP_08006103, MEROPS ID: MER0194920;
SEQ ID NO: 24: Bacillus sp. M28 family peptidase amino acid sequence ZP_09358807, MEROPS ID: MER0194188;
SEQ ID NO: 25: Bacillus sp. M28 family peptidase amino acid sequence YP_006233740, MEROPS ID: MER0194362;
SEQ ID NO: 26: Bacillus subtilis M28 family peptidase amino acid sequence, MEROPS ID: MER1247048;
SEQ ID NO: 27: Bacillus cereus M28 family peptidase amino acid sequence, MEROPS ID: MER0023520;
SEQ ID NO: 28: Bacillus cereus M28 family peptidase amino acid sequence YP_002445872, MEROPS ID: MER0053910;
SEQ ID NO: 29: Bacillus cereus M28 family peptidase amino acid sequence ZP 04215035, MEROPS ID: MER0190082;
SEQ ID NO: 30: Bacillus cereus M28 family peptidase amino acid sequence ZP 04200462, MEROPS ID: MER0190228;
SEQ ID NO: 31: Bacillus cereus M28 family peptidase amino acid sequence EJP89784, MEROPS ID: MER0321040;
SEQ ID NO: 32: Bacillus cereus M28 family peptidase amino acid sequence, MEROPS ID: MER0493011;
SEQ ID NO: 33: Bacillus cereus M28 family peptidase amino acid sequence, MEROPS ID: MER0493016;
SEQ ID NO: 34: Bacillus subtilis M28 family peptidase amino acid sequence YP_004205228, MEROPS ID: MER0238461; SEQ ID NO: 35: Bacillus thuringiensis M28 family peptidase amino acid sequence, MEROPS ID: MER0023524;
SEQ ID NO: 36: Bacillus thuringiensis M28 family peptidase amino acid sequence ZP_04093367, MEROPS ID: MER0039838;
SEQ ID NO: 37: Bacillus thuringiensis M28 family peptidase amino acid sequence ZP_04075003, MEROPS ID: MER0190208;
SEQ ID NO: 38: Bacillus thuringiensis M28 family peptidase amino acid sequence, MEROPS ID: MER0190209;
SEQ ID NO: 39: Bacillus thuringiensis M28 family peptidase amino acid sequence, MEROPS ID: MER0190212;
SEQ ID NO: 40: Bacillus thuringiensis M28 family peptidase amino acid sequence ZP_04105792, MEROPS ID: MER0190217;
SEQ ID NO: 41: Bacillus thuringiensis M28 family peptidase amino acid sequence, MEROPS ID: MER0243989;
SEQ ID NO: 42: Bacillus thuringiensis M28 family peptidase amino acid sequence, MEROPS ID: MER0244164;
SEQ ID NO: 43: Bacillus thuringiensis M28 family peptidase amino acid sequence, MEROPS ID: MER0493028;
SEQ ID NO: 44: Brevibacillus sp. M28 family peptidase amino acid sequence ZP 10575762, MEROPS ID: MER0230906;
SEQ ID NO: 45: Brevibacillus sp. M28 family peptidase amino acid sequence ZP 10742386, MEROPS ID: MER0271989;
SEQ ID NO: 46: Flavobacterium sp. M28 family peptidase amino acid sequence ZP_10482807, MEROPS ID: MER0185182;
SEQ ID NO: 47: Streptomyces sp. M28 family peptidase amino acid sequence EFL19287, MEROPS ID: MER0195352;
SEQ ID NO: 48: Bacillus licheniformis truncated M28 family peptidase nucleotide sequence;
SEQ ID NO: 49: Bacillus licheniformis truncated M28 family peptidase amino acid sequence;
SEQ ID NO: 50: oPlastBug-361 oligonucleotide
SEQ ID NO: 51: oPlastBug-362 oligonucleotide
SEQ ID NO: 52: Bacillus licheniformis mature M28 family peptidase amino acid sequence with Yarrowia lipolytica LIP2 signal peptide;
SEQ ID NO: 53: Nucleotide sequence of Bacillus licheniformis mature M28 family peptidase amino acid sequence with Yarrowia lipolytica LIP2 signal peptide optimised to Yarrowia lipolytica;
SEQ ID NO: 54: truncated Bacillus licheniformis mature M28 family peptidase amino acid sequence with Yarrowia lipolytica LIP2 signal peptide;
SEQ ID NO: 55: Nucleotide sequence of truncated Bacillus licheniformis mature M28 family peptidase amino acid sequence with Yarrowia lipolytica LIP2 signal peptide optimised to Yarrowia lipolytica;
SEQ ID NO: 56: oPlastBug-363 oligonucleotide
SEQ ID NO: 57: oPlastBug-364 oligonucleotide

### DETAILED DESCRIPTION OF THE INVENTION

The present invention concerns a method of degrading a hydrocarbon chain such as a polyolefin and/or a polystyrene, wherein a specific enzyme or micro-organism of the present invention is used for degrading said hydrocarbon chain. In one embodiment of the present invention a polyolefin and/or a polystyrene or a material comprising one or more polyolefins and/or polystyrenes or types of polyolefins and/or polystyrenes (such as plastics or polymers of fossil origin, bio-based polymers or plastic material, polymer composites, copolymers, packaging material, textile, plastics or synthetic polymers (e.g. oil-based and/or biobased) containing waste material) is allowed to contact with an enzyme or micro-organism capable of degrading the polyolefin(s) and/or the polystyrene(s). In one embodiment of the invention the material comprising one or more polyolefins and/or polystyrenes or types of polyolefins and/or polystyrenes is a recycled material or from a recycled material.

As used herein, “a plastic” refers to a material comprising or consisting of synthetic and/or semi-synthetic organic compounds and having the capability of being molded or shaped. As used herein “a synthetic polymer” refers to a human-made polymer. Synthetic polymers can be classified into four main categories: thermoplastics, thermosets, elastomers, and synthetic fibers. Thermoplastics are a type of synthetic polymers that become moldable and malleable past a certain temperature, and they solidify upon cooling. Thermosets become hard and cannot change shape once they have set. Elastomers are flexible polymers. Synthetic fibers are fibers made by humans through a chemical synthesis.

As used herein ‘polyolefin’ refers to a type of polymer produced from a simple olefin (e.g., called an alkene with the general formula CₙH₂ₙ) as a monomer. For example, polyethylene and polypropylene are common polyolefins. Depending on a polymerization method utilized for producing a polyolefin, the polyolefin hydrocarbon chain can sometimes comprise a specific group or groups such as a ketone group e.g. at the end of the chain. Polyolefins can be non-toxic, non-contaminating and lighter than water. In one embodiment the polyolefin is polyethylene (PE), cross-linked polyethylene (PEX or XLPE), ultra-high molecular weight polyethylene (UHMWPE), high-density polyethylene (HDPE), medium-density polyethylene (MDPE), linear low-density polyethylene (LLDPE), low density polyethylene (LDPE), very low-density polyethylene (VLDPE), polypropylene (PP), polymethylpentene (PMP), polybutene-1 (PB-1), polyisobutylene (PIB), or any combination thereof. In one embodiment the polyolefin is polyethylene, polypropylene or a combination thereof.

Polyethylene (PE) (formula (C₂H₄)ₙ) consists of long chain polymers of ethylene and it can be produced as high-density (HDPE), medium-density (MDPE), or low-density polyethylene (LDPE). PE can be chemically synthesized by polymerization of ethane and it is highly variable since side chains can be obtained depending on the manufacturing process. LDPE has more branching than HDPE (i.e. has a high degree of short- and long-chain branching), and therefore it's intermolecular forces are weaker, its tensile strength is lower, and its resilience is higher. Also, because its molecules are less tightly packed and less crystalline due to the side branches, its density is lower. In one embodiment LDPE is defined by a density range of about 910-930 kg/m³, MDPE is defined by a density range of about 926 to 0.940 kg/m³, and/or the density range of HDPE is about 930 to 970 kg/m³.

Cross-linked polyethylene (PEX or XLPE) is a form of polyethylene with cross-linked bonds in the polymer structure, changing the thermoplastic to a thermoset. Indeed, crosslinking enhances the temperature properties of the base polymer and furthermore e.g. tensile strength, scratch resistance, and resistance to brittle fracture.

Ultra-high molecular weight polyethylene (UHMWPE) is a thermoplastic, and it is made up of extremely long chains of PE, which all align in the same direction. The extremely long chain can usually have a molecular mass between 3.5 and 7.5 million amu.

Linear low-density polyethylene (LLDPE) is a substantially linear PE with significant numbers of short branches. LLDPE differs structurally from conventional LDPE because of the absence of long chain branching.

Very low density polyethylene (VLDPE) is a type of LLDPE with higher levels of short-chain branches than standard LLDPE. VLDPE can be defined e.g. by a density range of 0.880-0.910 g/cm³.

Polypropylene (PP) (formula (C₃H₆)ₙ) is a thermoplastic, which can be produced e.g. via chain-growth polymerization from the monomer propylene. PP is partially crystalline and non-polar. Its properties are very similar to PE, but it is e.g. slightly harder and more heat resistant.

Polymethylpentene (PMP) (i.e. poly(4-methyl-1-pentene), formula (C₆H₁₂)ₙ) is a thermoplastic polymer of 4-methyl-1-pentene. It is a high-molecular weight hydrocarbon and an extremely low density olefinic commodity thermoplastic. PMP's chemical resistance is close to that of PP. Compared to PP it is more easily softened by unsaturated and aromatic hydrocarbons, and chlorinated solvents, and slightly more susceptible to attack by oxidizing agents.

Polybutene-1 (PB-1) (formula (C₄H₈)ₙ) is a high molecular weight, linear, isotactic, and semi-crystalline polymer. Polybutylene can be produced by polymerization of 1-butene using supported Ziegler-Netta catalysts.

Polyisobutylene (PIB) (formula (C₄H₈)ₙ) can be prepared by polymerization of isobutene. The molecular weight of the PIB can determine the application. For example, low MW PIBs can be used as plasticizers, and medium and high MW PIBs in adhesives.

As used herein ‘polystyrene’ (PS) refers to a synthetic, thermoplastic polymer produced from an aromatic hydrocarbon styrene. Polystyrene can be solid or foamed. Under ASTM (American Society for testing and Materials) standards, polystyrene is regarded as not biodegradable.

In one embodiment of the invention the enzyme capable of degrading a polyolefin and/or a polystyrene, or a polyolefin and/or a polystyrene containing material is from a bacterium (gram-positive or gram-negative) or fungus, and/or the micro-organism capable of degrading a polyolefin and/or a polystyrene or a polyolefin and/or polystyrene containing material is a bacterium (gram-positive or gram-negative) or fungus. As used herein “fungus”, “fungi” and “fungal” refer to yeast and filamentous fungi (i.e., moulds). In one embodiment of the invention the fungus is a yeast or filamentous fungus.

As used herein, “degradation” of a polyolefin and/or a polystyrene, plastic, synthetic or non-synthetic polymer refers to either partial or complete degradation of a polyolefin and/or a polystyrene, plastic, synthetic or non-synthetic polymer to a shorter hydrocarbon chain (such as a hydrocarbon chain comprising one or more organic compounds, a long ketone, a long alcohol, a long fatty acid), oligomers and/or monomers. The degradation also encompasses modification of a polyolefin and/or a polystyrene with addition of oxygen, hydroxy-, aldehyde- and/or carboxy-group(s) at or in the proximity of the cleavage site, or to the middle of the carbon chain. The degradation also encompasses modification of a polyolefin and/or a polystyrene by generation of double bonds to the carbon chain. Said degradation can also include lowering of the molecular weight of a polyolefin and/or a polystyrene, lowering of the average molecular weight, lowering of the molar mass in the peak of maximum and/or increase in polydispersity of a polyolefin and/or a polystyrene. Indeed, any loss in the chain length of a polyolefin and/or a polystyrene can e.g., lower tensile strength. “Enzymatic or microbial degradation” refers to a degradation caused by an enzyme or micro-organism, respectively. According to some hypothesis, in the microbial degradation the larger polymers are initially degraded by secreted exoenzymes or by outer membrane bound enzymes into smaller subunits (different length oligomers) that can be incorporated into the cells of micro-organisms and further degraded through the classical degradation pathways to yield energy and/or suit as building blocks for catabolismor metabolism.

Many plastics or other materials are mixtures comprising synthetic or semi-synthetic polymers and furthermore solubilizers and optionally other chemical agents for altering the mechanical and physical properties of said plastics or materials. For example, the plastic material may contain an additive, which increases the hydrophilicity of the plastic and makes it more prone to the enzymatic degradation. The solubilizers and other chemical compounds may also be targets of enzymatic or microbial biodegradation.

In one embodiment of the invention the enzyme or a fragment thereof, micro-organism or host cell comprises polyolefin and/or polystyrene degrading activity.

In one embodiment of the invention the enzyme or a fragment thereof, micro-organism or host cell comprises polyolefin, PE, PEX, UHMWPE, HDPE, MDPE, LLDPE, LDPE, VLDPE, PP, PMP, PB-1, or PIB degrading activity, or any combination thereof; and/or polystyrene degrading activity; or is capable of degrading a polyethylene and/or a polypropylene and/or polystyrene. In one embodiment the enzymes, fragments, micro-organisms or host cells of the present invention can be capable of utilizing short, medium-sized and/or long hydrocarbon chain polyolefins and/or polystyrenes (such as those having a molecular weight of 100 Da-50 000 kDa, e.g. 5 000 Da-10 000 kDa).

Degradation of a polyolefin and/or a polystyrene, a material comprising a polyolefin and/or a polystyrene, synthetic polymer or plastic can result in at least one or more degradation products. In one embodiment of the invention, at least one or more degradation products selected from the group consisting of an alkane, alkene, alkyne, cycloalkane, alkadiene, ketone (e.g. ketone C2-C32), fatty acid, alcohol, aldehyde, epoxy, benzene, styrene, diacid, 2-decanone, 2-dodecanone, 2-tetradecanone, 2-hexadecanone, 2-heptadecanone and 2-dotriacontanone are obtained or obtainable by the degradation of the hydrocarbon chain. For example, PE can be degraded to an alkane, alkene, alkyne, cycloalkane, alkadiene, ketone (e.g. ketone C2-C32), fatty acid, alcohol, aldehyde, diacid, 2-decanone, 2-dodecanone, 2-tetradecanone, 2-hexadecanone, 2-heptadecanone and/or 2-dotriacontanone. And for example, PP can be degraded to an alkane, alkene, alkyne, cycloalkane, alkadiene, ketone (e.g. ketone C2-C32), fatty acid, alcohol, aldehyde, and/or diacid. Also PS can be degraded to styrene, styrene oxide, α-methyl styrene, toluene, benzoic acid, ethylbenzene, cumyl alcohol, acetophenone, benzaldehyde, benzoic acid, 1-phenylethanol, 2-phenylethanol, 1-phenyl-1,2,3,8a-tetrahydronaphthalene, 1,1-diphenyl ethylene, 2,4-diphenyl-1-butene, trans-1,2-diphenylcyclobutane, 1,3-diphenylbutene-3, cis-1,2-diphenylcyclobutane, 1-phenyltetralin, 2-phenyltetralin, 1,2-diphenylethane-1,2-dione, (3-phenylbut-3-enyl)benzene, (Z)-1,4-diphenyl-1-butene, 2,4,6-triphenyl-1-hexene, 1,3,5-triphenylcyclohexane, 1,3,5-Triphenylhexene-5, 1-phenyl-4-(1-phenylethyl)tetralin, (1-methyl-2,2-diphenylcyclopropyl) sulfanylbenzene, 2,4-di-tert-butylphenol, 2-ethyl-1-hexanol, tris(2,4-ditert-butylphenyl) phosphite, 1,4,7-trioxacyclotridecane-8,13-dione, 1,6-dioxacyclododecane-7,12-dione, 2,6-di-tert-butyl-p-benzoquinone, and/or 7,9-Di-tert-butyl-1-oxaspiro[4.5] deca-6,9-diene-2,8-dione.

In one embodiment of the invention only the enzyme(s) or micro-organism(s) or a combination thereof is (are) needed for a biotechnical or enzymatic degradation of a polyolefin and/or a polystyrene, a combination of different types of polyolefins and/or polystyrenes. In other words, no other degradation methods such as UV light or mechanical disruption or chemical degradation are needed in said embodiment. In other embodiments, biotechnical, enzymatic or microbial degradations can be combined with each other, i.e., in addition to the enzyme(s) or micro-organism(s) of the present invention, another enzyme(s) or micro-organism(s) can produce specific products from the degradation products or can degrade them further. In other embodiments, biotechnical, enzymatic or microbial degradation can be combined with one or more other degradation methods (e.g., non-enzymatic degradation methods) including but not limited to UV light, gamma irradiation, microwave treatment, mechanical disruption and/or chemical degradation. In one embodiment of the invention the method of degrading a polyolefin and/or a polystyrene is a biotechnical method, or the method comprises degradation of the polyolefin and/or polystyrene by non-enzymatic methods or means. Non-enzymatic, non-microbial or non-biotechnical degradation methods or steps including pretreatments can be carried out sequentially (e.g., before or after) or simultaneously with the biotechnical, microbial or enzymatic degradation. For example, the polyolefin and/or polystyrene can be oxidized using e.g. oxides, such as hydrogen peroxide or ZnO, in order to make the hydrocarbon more hydrophilic and thus more prone to the enzymatic degradation. In addition, solvents can be used for separating polymer chains from each other before enzymatic degradation of a polyolefin and/or polystyrene. One or more (pre) treatments with solvents enable micro-organisms, enzymes or fragments thereof to access and degrade polyolefin and/or polystyrene in the inner parts of the plastic material to be degraded. Suitable solvents for plastics or polyolefins and/or polystyrenes include but are not limited to toluene, xylene, benzene, trichlorobenzene, trichloroethylene, and/or tetralin.

In one embodiment the present invention concerns an isolated enzyme or a fragment thereof belonging to M28F peptidase subfamily, wherein said enzyme or fragment is capable of degrading a polyolefin and/or a polystyrene, and/or having at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, wherein said enzyme or fragment is capable of degrading a polyolefin and/or a polystyrene.

In one embodiment the present invention concerns an isolated enzyme or a fragment thereof belonging to M28F peptidase subfamily and having at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, wherein said enzyme or fragment is capable of degrading a polyolefin and/or a polystyrene.

In one embodiment the present invention concerns an isolated enzyme or a fragment thereof belonging to M28F peptidase subfamily and comprising one or more amino acids selected from the group comprising or consisting of Gly264, Ala265, Ser270, Gly271 and Gly305 corresponding to the amino acid positions presented in SEQ ID NO: 4, wherein said enzyme or fragment is capable of degrading a polyolefin and/or polystyrene, and/or having at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, wherein said enzyme or fragment is capable of degrading a polyolefin and/or a polystyrene.

In one embodiment, the present invention relates to an isolated enzyme or a fragment thereof belonging to M28F peptidase subfamily and comprising amino acids Gly264, Ala265, Ser270, Gly271 and Gly305 corresponding to the amino acid positions presented in SEQ ID NO: 4, wherein said enzyme or fragment is capable of degrading a polyolefin and/or polystyrene, and/or having at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, wherein said enzyme or fragment is capable of degrading a polyolefin and/or a polystyrene.

In one embodiment the isolated enzyme is a truncated enzyme belonging to M28F peptidase subfamily, wherein said truncated enzyme is capable of degrading a polyolefin and/or a polystyrene.

As used herein, an enzyme belonging to the M28F peptidase subfamily refers to an enzyme classified according to the catalytic type, family and subfamily from the Peptidase database MEROPS (https://www.ebi.ac.uk/MEROPS/). Functional domains are verified according to Pfam (http://pfam.xfam.org/), InterPro (http://www.ebi.ac.uk/interpro/) and UniProt (https://www.uniprot.org/) databases. The subfamily M28F of M28 family peptidases contains aminopeptidases and carboxypeptidases. They belong to clan MH of metalloproteases and include YwaD peptidases (MEROPS-ID M28.009), AM-1 aminopeptidases (MEROPS-ID M28.020), unassigned peptidases (MEROPS-ID M28.UPF) and non-peptidase homologues (MEROPS-ID M28.UNF).

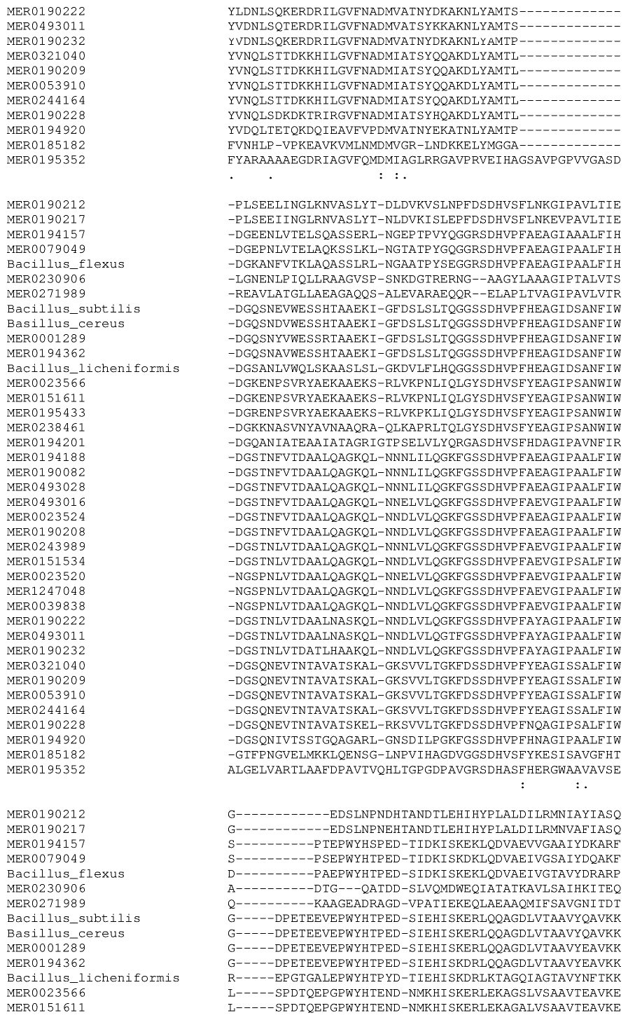

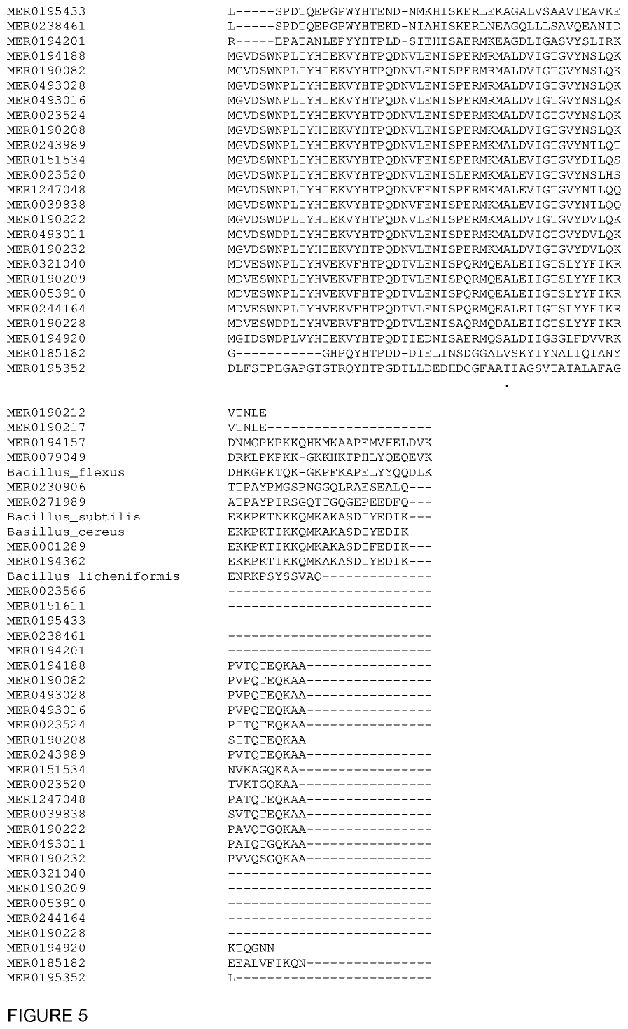

As used herein, a truncated enzyme belonging to M28F peptidase subfamily refers to a wild-type enzyme missing the protease associated (PA) domain or a genetically modified enzyme where the protease associated (PA) domain is removed using methods known to a person skilled in the art. The alignment shown in FIG. 5 presents also sequences of the truncated enzymes belonging to M28F peptidase subfamily. In one embodiment, the truncated enzyme belonging to M28F peptidase subfamily comprises amino acids Gly264, Ala265, Ser270, Gly271 and Gly305 corresponding to the amino acid positions presented in SEQ ID NO: 4 but is missing the amino acids 101-225 corresponding to SEQ ID NO: 4.

In one embodiment, the present invention relates to a truncated enzyme or a fragment thereof belonging to M28F peptidase subfamily and comprising one or more amino acids selected from the group comprising or consisting of amino acids Gly264, Ala265, Ser270, Gly271 and Gly305 corresponding to the amino acid positions presented in SEQ ID NO: 4, wherein said truncated enzyme or fragment is capable of degrading a polyolefin and/or polystyrene, and/or having at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 49, wherein said truncated enzyme or fragment is capable of degrading a polyolefin and/or a polystyrene.

In one embodiment, the present invention relates to a truncated enzyme or a fragment thereof belonging to M28F peptidase subfamily and comprising amino acids Gly264, Ala265, Ser270, Gly271 and Gly305 corresponding to the amino acid positions presented in SEQ ID NO: 4, wherein said truncated enzyme or fragment is capable of degrading a polyolefin and/or polystyrene, and/or having at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 49, wherein said truncated enzyme or fragment is capable of degrading a polyolefin and/or a polystyrene.

Also, the present invention concerns a micro-organism or a host cell comprising an enzyme or a fragment thereof belonging to M28F peptidase subfamily, wherein said enzyme or fragment is capable of degrading a polyolefin and/or polystyrene and/or having at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, wherein said enzyme or fragment is capable of degrading a hydrocarbon chain or a polyolefin.

In one embodiment, the present invention relates to a micro-organism or a host cell comprising an isolated enzyme or a fragment thereof belonging to M28F peptidase subfamily and having at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, wherein said enzyme or fragment is capable of degrading a polyolefin and/or a polystyrene.

In one embodiment, the present invention relates to a micro-organism or a host cell comprising an isolated enzyme or a fragment thereof belonging to M28F peptidase subfamily and comprising one or more amino acids selected from the group comprising or consisting of Gly264, Ala265, Ser270, Gly271 and Gly305 corresponding to the amino acid positions presented in SEQ ID NO: 4, wherein said enzyme or fragment is capable of degrading a polyolefin and/or polystyrene, and/or having at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, wherein said enzyme or fragment is capable of degrading a polyolefin and/or a polystyrene.

In one embodiment, the present invention relates to a micro-organism or a host cell comprising an isolated enzyme or a fragment thereof belonging to M28F peptidase subfamily and comprising the amino acids Gly264, Ala265, Ser270, Gly271 and Gly305 corresponding to the amino acid positions presented in SEQ ID NO: 4, wherein said enzyme or fragment is capable of degrading a polyolefin and/or polystyrene, and/or having at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, wherein said enzyme or fragment is capable of degrading a polyolefin and/or a polystyrene.

Said relevant or specific amino acids can be e.g. consensus or conserved amino acids. As used herein “a consensus amino acid” refers to an amino acid which is the one occurring most frequently at that amino acid site in the different sequences e.g. across species. As used herein “conserved amino acids” refers to identical or similar amino acids in polypeptides or proteins across species. Conservation indicates that an amino acid has been maintained by natural selection.

The enzyme of the present invention refers to not only fungal or bacterial but also any other enzyme homologue from any micro-organism, organism or mammal. Also, all isozymes, isoforms and variants are included with the scope of said enzyme. In one embodiment of the method, enzyme, fragment, micro-organism or host cell of the present invention, the enzyme originates from or is an enzyme of a bacterium or fungus selected from the group comprising or consisting of Bacillus, Paenibacillus, Achromobacter, Acinetobacter, Alcanivorax, Aneurinibacillus, Arthrobacter, Aspergillus, Brevibacillus, Brucella, Chitinophaga, Citrobacter, Comamonas, Cordyceps, Cupriavidus, Delftia, Enterobacter, Escherichia, Exiguobacterium, Flavobacterium, Fusarium, Halomonas, Hyphomicrobium, Klebsiella, Kocuria, Leucobacter, Lysinibacillus, Macrococcus, Methylobacterium, Methylocella, Microbacterium, Micrococcus, Moraxella, Mucor, Nesiotobacter, Nocardia, Ochrobactrum, Pantoea, Paracoccus, Penicillium, Pleurotus, Pseudomonas, Rahnella, Ralstonia, Rhizobium, Rhodococcus, Serratia, Sphingobacterium, Staphylococcus, Stenotrophomonas, Streptococcus, Streptomyces, Trichoderma, Vibrio, Virgibacillus and Xanthobacter; or the enzyme is an enzyme of a bacterium or fungus selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp, Aspergillus awamori, Aspergillus flavus, Aspergillus fumigatus, Aspergillus glaucus, Aspergillus niger, Aspergillus oryzae, Aspergillus sp. Aspergillus sydowii, Aspergillus terreus, Bacillus amyloliquefaciens, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Brevibacillus sp., Brucella anthropi, Chitinophaga sp., Citrobacter amalonaticus, Comamonas sp., Cordyceps confragosa, Cupriavidus necator, Delftia sp., Delftia tsuruhatensis, Enterobacter asburiae, Enterobacter sp., Escherichia coli, Exiguobacterium sp. Flavobacterium sp., Flavobacterium petrolei, Flavobacterium pectinovorum, Flavobacterium aquicola, Fusarium solani, Fusarium sp., Halomonas venusta, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus luteus, Micrococcus lylae, Moraxella sp., Mucor circinelloides, Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Penicillium chrysogenum, Penicillium oxalicum, Penicillium ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Sphingobacterium multivorum, Staphylococcus epidermidis, Staphylococcus cohnii, Staphylococcus sp., Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Vibrio alginolyticus, Vibrio parahaemolyticus, Virgibacillus halodenitrificans, Xanthobacter autotrophicus, and Xanthobacter tagetidis.

In one embodiment “an enzyme of a bacterium or fungus” refers to a situation, wherein the amino acid sequence of the enzyme has the same amino acid sequence as a wild type enzyme of a bacterium or fungus (e.g. any of the above listed bacteria or fungus) or the amino acid sequence of the enzyme has a high sequence identity (e.g. 50% or more, 60% or more, 70% or more, 80% or more, 85% or more, 90% or more, or 95% or more) to an amino acid sequence of a wild type bacterial or fungal enzyme (e.g. of any of the above listed bacteria or fungus). In other words, the amino acid sequence of the enzyme used in the present invention can be modified (e.g. genetically modified).

In one embodiment, the enzyme, fragment, micro-organism or host cell is a genetically modified enzyme, fragment, micro-organism or host cell. In a specific embodiment the enzyme, fragment, micro-organism or host cell has an increased ability to degrade a hydrocarbon chain or a polyolefin compared to the corresponding unmodified enzyme, fragment, micro-organism or host cell, respectively. In one embodiment the enzyme, micro-organism or host cell comprises a genetic modification increasing an enzyme activity or the amount of a specific enzyme in a micro-organism or host cell. Genetic modifications (e.g. resulting in increased enzyme activity, increased expression of an enzyme, or increased or faster degradation of a hydrocarbon chain) include but are not limited to genetic insertions, deletions, disruptions or substitutions of one or more genes or a fragment(s) thereof or insertions, deletions, disruptions or substitutions of one or more nucleotides (e.g. insertion of a polynucleotide encoding an enzyme), or addition of plasmids. For example, one or several polynucleotides encoding an enzyme of interest can be integrated to the genome of a micro-organism or host cell. As used herein “disruption” refers to insertion of one or several nucleotides into a gene or polynucleotide sequence resulting in a lack of the corresponding polypeptide or enzyme or presence of non-functional polypeptide or enzyme with lowered activity. Methods for making any genetic modifications or modifying micro-organisms or host cells (e.g. by adaptive evolution strategy) are generally well known by a person skilled in the art and are described in various practical manuals describing laboratory molecular techniques.

In one embodiment the enzyme or a fragment thereof has one or more genetic modifications (e.g. a targeted mutation or a modification by an adaptive evolution) after one or more amino acids corresponding to the amino acids selected from the group comprising or consisting of Gly264, Ala265, Ser270, Gly271 and Gly305 presented in SEQ ID NO: 4. As used herein “after one or more amino acids” refers to immediately after said amino acid(s) e.g. a modification at least in the next amino acid or later after said amino acid (e.g. 1-50 amino acids, 1-30 amino acids, 1-20 amino acids, 1-10 amino acids or 1-5 amino acids after the specific amino acid mentioned above in the list of this paragraph).

As used herein “increased degradation (activity/ability/capability) of a polyolefin and/or a polystyrene” or “faster degradation (activity/ability/capability) of a polyolefin and/or a polystyrene” of an enzyme or micro-organism refers to the presence of higher activity or more activity of an enzyme or micro-organism, when compared to another enzyme or micro-organism, e.g. a genetically unmodified (wild type) enzyme or micro-organism. “Increased or faster degradation” may result e.g. from the presence of a specific enzyme in a micro-organism or an up-regulated gene or polypeptide expression in a micro-organism or an increased secretion of an enzyme by a micro-organism. Also, “increased or faster degradation” may result e.g. from the presence of (enhancing) mutations of a specific enzyme having degradation capability.

As used herein “up-regulation of the gene or polypeptide expression” refers to excessive expression of a gene or polypeptide by producing more products (e.g. mRNA or polypeptide, respectively) than an unmodified micro-organism. For example, one or more copies of a gene or genes may be transformed to a cell (e.g. to be integrated to the genome of the cell) for upregulated gene expression. The term also encompasses embodiments, where a regulating region such as a promoter or promoter region has been modified or changed or a regulating region (e.g. a promoter) not naturally present in the micro-organism has been inserted to allow the overexpression of a gene. Also, epigenetic modifications such as reducing DNA methylation or histone modifications as well as classical mutagenesis are included in “genetic modifications”, which can result in an upregulated expression of a gene or polypeptide. As used herein “increased or up-regulated expression” refers to an increased expression of the gene or polypeptide of interest compared to a wild type micro-organism without the genetic modification. Expression or increased expression can be proved for example by western, northern or southern blotting or quantitative PCR or any other suitable method known to a person skilled in the art. As used herein “increased secretion of an enzyme by a micro-organism” refers to a secretion of an enzyme outside of a cell, which produces said enzyme. Increased secretion may be caused e.g. by an increased or up-regulated expression of the gene or polypeptide of interest or by improved secretion pathway of the cell or molecules participating in the secretion of said enzyme. In one embodiment secretion of an enzyme can be increased by adding one or more glycosylation sites to the enzyme or by altering or deleting one or more glycosylation sites.

In one embodiment the genetically modified enzyme, micro-organism, host cell or polynucleotide is a recombinant enzyme, micro-organism, host cell or polynucleotide. As used herein, “a recombinant enzyme, micro-organism, host cell or polynucleotide” refers to any enzyme, micro-organism, host cell or polynucleotide that has been genetically modified to contain different genetic material compared to the enzyme, micro-organism, host cell or polynucleotide before modification (e.g. comprise a deletion, substitution, disruption or insertion of one or more nucleic acids or amino acids e.g. including an entire gene(s) or parts thereof). The recombinant micro-organism or host cell may also contain other genetic modifications than those specifically mentioned or described in the present disclosure. Indeed, the micro-organism or host cell may be genetically modified to produce, not to produce, increase production or decrease production of e.g. other polynucleotides, polypeptides, enzymes or compounds than those specifically mentioned in the present disclosure. In certain embodiments, the genetically modified micro-organism or host cell includes a heterologous polynucleotide or enzyme. The micro-organism or host cell can be genetically modified by transforming it with a heterologous polynucleotide sequence that encodes a heterologous polypeptide. For example, a cell may be transformed with a heterologous polynucleotide encoding an enzyme of the present invention either without a signal sequence or with a signal sequence. Alternatively, for example heterologous promoters or other regulating sequences can be utilized in the micro-organisms, host cells or polynucleotides of the invention. As used herein “a heterologous polynucleotide or enzyme” refers to a polynucleotide or enzyme, which does not naturally occur in a cell or micro-organism. In one embodiment of the present invention, the enzyme or fragment thereof is encoded by a heterologous polynucleotide sequence and optionally expressed by a micro-organism or host cell.

Genetic modifications may be carried out using conventional molecular biological methods. Genetic modification (e.g. of an enzyme or micro-organism) can be accomplished in one or more steps via the design and construction of appropriate vectors and transformation of the micro-organism cell with those vectors. For example, electroporation, protoplast-PEG and/or chemical (such as calcium chloride or lithium acetate based) transformation methods can be used. Also, any commercial transformation methods are appropriate. Suitable transformation methods are well known to a person skilled in the art.

The term “vector” refers to a nucleic acid compound and/or composition that transduces, transforms, or infects a micro-organism or a host cell, thereby causing the cell to express polynucleotides and/or proteins other than those native to the cell, or in a manner not native to the cell. An “expression vector” contains a sequence of nucleic acids to be expressed by the modified micro-organism. Optionally, the expression vector also comprises materials to aid in achieving entry of the nucleic acids into the micro-organism, such as a virus, liposome, protein coating, or the like. The expression vectors contemplated for use in the present invention include those into which a nucleic acid sequence (i.e. polynucleotide) can be inserted, along with any preferred or required operational elements. Further, the expression vector must be one that can be transferred into a micro-organism or host cell and replicated therein. Vectors can be circularized or linearized and may contain restriction sites of various types for linearization or fragmentation. In specific embodiments expression vectors are plasmids, particularly those with restriction sites that have been well documented and that contain the operational elements preferred or required for transcription of the nucleic acid sequence. Such plasmids, as well as other expression vectors, are well known to those of ordinary skill in the art. Useful vectors may for example be conveniently obtained from commercially available micro-organism, yeast or bacterial vectors. Successful transformants can be selected using the attributes contributed by the marker or selection gene. Screening can be performed e.g. by PCR or Southern analysis to confirm that the desired genetic modifications (e.g. deletions, substitutions or insertions) have taken place, to confirm copy number or to identify the point of integration of nucleic acids (i.e. polynucleotides) or genes into the micro-organism cell's genome.

Indeed, the present invention also relates to a polynucleotide encoding the enzyme of the present invention or a fragment thereof, and an expression vector or plasmid comprising said polynucleotide of the present invention.

In a specific embodiment the enzyme of the present invention comprises or has a sequence having at least 15%, 20%, 25%, 30%, 35%, 40%, 45%, 50%, 55%, 60%, 65%, 70%, 71%, 72%, 73%, 74%, 75%, 76%, 77%, 78%, 79%, 80%, 80.5%, 81%, 81.5%, 82%, 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99% (e.g. 99.1%, 99.2%, 99.3%, 99.4%, 99.5%, 99.6%, 99.7%, 99.8% or 99.9%) or 100% sequence identity to SEQ ID NO: 2, 4, 6, 8 or 49, or an enzymatically active fragment or variant thereof. Said enzyme can be genetically modified (i.e. differs from the wild type enzyme) or unmodified. In a specific embodiment an enzyme is an isolated enzyme.

In one embodiment of the invention the enzyme has at least 15, 20, 25, 26, 27, 28, 29, 30, 35, 40, 45, 50, 55, 60, 65, 70, 71, 72, 73, 74, 75, 76, 77, 78, 79, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99 (e.g. 99.1, 99.2, 99.3, 99.4, 99.5, 99.6, 99.7, 99.8 or 99.9%), or 100% sequence identity to SEQ ID NO: 4 (SEQ ID NO: 4 is a Bacillus licheniformis M28 family peptidase amino acid sequence).

In one embodiment of the invention the enzyme has at least 15, 20, 25, 26, 27, 28, 29, 30, 35, 40, 45, 50, 55, 60, 65, 70, 71, 72, 73, 74, 75, 76, 77, 78, 79, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99 (e.g. 99.1, 99.2, 99.3, 99.4, 99.5, 99.6, 99.7, 99.8 or 99.9%), or 100% sequence identity to SEQ ID NO: 49 (SEQ ID NO: 49 is a Bacillus licheniformis M28 family peptidase amino acid sequence wherein the amino acids of the PA domain (amino acids 101-225 of SEQ ID NO: 4) are missing or removed.

In one embodiment, the enzyme or fragment comprises a signal sequence, e.g. a heterologous signal sequence or a signal sequence of an exogenous host cell producing said enzyme of a fragment thereof. The signal sequence can be located e.g. after or before the amino acid sequence of the enzyme e.g. for secreting said enzyme outside of the cell. The signal sequence can be any signal sequence i.e. a short polypeptide present at the N-terminus of synthesized polypeptides that are destined towards the secretory pathway, said polypeptides including but not limited to those polypeptides that are targeted inside specific organelles, secreted from the cell, or inserted into cellular membranes. In one embodiment the enzyme or fragment thereof comprises a signal sequence, does not comprise a detectable signal sequence, is secreted out of the cell which produces it, and/or is not secreted out of the cell which produces it. In one embodiment the enzyme or fragment thereof does not comprise a detectable signal sequence and is secreted out of the cell which produces it.

A polynucleotide of the present invention encodes the enzyme of the present invention or a fragment thereof. In a specific embodiment the polynucleotide comprises a sequence having a sequence identity of at least 15%, 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% to SEQ ID NO: 1, 3, 5, 7 or 48 or a variant thereof. Said polynucleotide can be genetically modified (i.e. differs from the wild type polynucleotide) or unmodified. In a specific embodiment the polynucleotide is an isolated polynucleotide.

Identity of any sequence or fragments thereof compared to the sequence of this disclosure refers to the identity of any sequence compared to the entire sequence of the present invention. As used herein, the % identity between the two sequences is a function of the number of identical positions shared by the sequences (i.e. % identity=#of identical positions/total #of positions×100), taking into account the number of gaps, and the length of each gap, which need to be introduced for optimal alignment of the two sequences. The comparison of sequences and determination of identity percentage between two sequences can be accomplished using mathematical algorithms available in the art. This applies to both amino acid and nucleic acid sequences. As an example, sequence identity may be determined by using BLAST (Basic Local Alignment Search Tools) or FASTA (FAST-All). In the searches, setting parameters “gap penalties” and “matrix” are typically selected as default. In one embodiment the sequence identity is determined against the full length sequence of the present disclosure.

Nucleic acid and amino acid databases (e.g., GenBank) can be used for identifying a polypeptide having an enzymatic activity or a polynucleotide sequence encoding said polypeptide. Sequence alignment software such as BLASTP (polypeptide), BLASTN (nucleotide) or FASTA can be used to compare various sequences. Briefly, any amino acid sequence having some homology to a polypeptide having enzymatic activity, or any nucleic acid sequence having some homology to a sequence encoding a polypeptide having enzymatic activity can be used as a query to search e.g. GenBank. Percent identity of sequences can conveniently be computed using BLAST software with default parameters. Sequences having an identities score and a positive score of a given percentage, using the BLAST algorithm with default parameters, are considered to be that percent identical or homologous.

For example, an enzyme comprising a polyolefin and/or polystyrene degrading activity and belonging to M28F peptidase subfamily and e.g., comprising amino acids Gly264, Ala265, Ser270, Gly271 and Gly305 corresponding to the amino acid positions presented in SEQ ID NO: 4, can be found as described in example 7. First, sequences originating from species which have been shown to degrade polyethylene, polypropylene or polystyrene can be collected from Peptidase database MEROPS (https://www.ebi.ac.uk/MEROPS: MEROPS:/) belonging to M28F subfamily. With the detected amino acid sequences or part of them or amino acid sequence(s) of previously known enzyme(s) sequence similarity searches against SEQ ID NO: 4 can be carried out e.g. by sequence alignment with ClustalW programme (https://www.genome.jp/tools-bin/clustalw) to detect corresponding consensus amino acids and their positions in amino acid sequence of interest. (See e.g. FIG. 5.)

In one embodiment, one or more of the amino acids Gly264, Ala265, Ser270, Gly271 and Gly305 (corresponding to the amino acid positions presented in SEQ ID NO: 4) are critical for the activity of the enzyme, e.g., degradation of a substrate.

The 3D and 2D structures of proteins showing the alfa helixes and beta sheets can used in predicting and finding the amino acids important for the activity of the protein. The position of critical amino acids can be localised from the predicted 2D and 3D structures as described in Example 8. According to the 3D and 2D structures amino acids Ser270, Gly271 and Gly305 were predicted to be located in alpha helixes 8 and 9. The 3D structure of the enzyme and positions of beta sheets and alfa helixes (2D structure) can be predicted e.g. with Phyre2 protein homology/analogy recognition engine V 2.0 (www.sbq.bio.ic.ac.uk/phyre2/html/page.cgi?id=index). The enzyme can comprise one or more specific amino acids or amino acid motifs for example affecting a hydrocarbon chain degrading activity (e.g. enabling different substrates and/or binding of metal ions).

In one embodiment of the method, enzyme, fragment, micro-organism or host cell of the present invention, the enzyme or fragment thereof comprises one or several amino acids selected from the group comprising or consisting of Gly264, Ala265, Ser270, Gly271 and Gly305, wherein the amino acids and positions correspond to the amino acids and positions presented in SEQ ID NO: 4. This means that the enzyme or fragment thereof comprises one or several amino acids, which correspond to the amino acids Gly264, Ala265, Ser270, Gly271 and Gly305 corresponding to the amino acid positions presented in SEQ ID NO: 4. In one embodiment the enzyme or fragment thereof comprises all amino acids selected from the group comprising or consisting of Gly264, Ala265, Ser270, Gly271 and Gly305, wherein the amino acids and positions correspond to the amino acids and positions presented in SEQ ID NO: 4.

In one embodiment one or more of the consensus amino acids affect the degrading activity (e.g. by increasing the degrading activity) of hydrocarbon chains or affect binding of a metal ion.

In one embodiment, the enzyme is capable of binding a divalent metal ion. In one embodiment the divalent metal ion is Zn2+, Cu2+, Ni2+, Mn2+, Fe2+, Mg2+, or any combination thereof. For example, the enzyme can bind at least Cu2+, Fe2+ or Mn2+; or Zn2+ and Cu2+. In one embodiment of the invention a divalent metal ion is part of the structure of the enzyme. In that case the enzyme cannot bind a divalent metal ion added to the culture.

In one embodiment the enzyme and/or micro-organism have been genetically modified and optionally have an increased ability to degrade a polyolefin or a polystyrene compared to the corresponding unmodified enzyme and/or micro-organism, respectively.

The presence, absence or amount of specific enzyme activities can be detected by any suitable method known in the art. Specific examples of studying enzyme activities of interest are well known to a person skilled in the art. Non-limiting examples of suitable detection methods include commercial kits on market, enzymatic assays, immunological detection methods (e.g., antibodies specific for said proteins), PCR based assays (e.g., qPCR, RT-PCR), and any combination thereof.

In one embodiment, the enzymes of the present invention have high turnover rates when degrading a polyolefin and/or a polystyrene, e.g. when compared to prior art enzymes. In specific embodiments the activity of an enzyme to degrade a polyolefin and/or a polystyrene is determined by an enzyme assay wherein said enzyme is allowed to contact with polyolefins and/or polystyrene (e.g. as described in example 2 and 9). In some embodiments the activity of an enzyme to degrade polyolefins and/or polystyrenes can be determined e.g. by detecting or measuring the degradation products of hydrocarbon chains polyolefins, alternatively or by analyzing the remaining starting material containing hydrocarbon chains or polyolefins after contacting the starting material with the enzymes.

Degradation of polyolefins and/or polystyrenes can be measured by any suitable method known in the field. In one embodiment polyolefins and/or polystyrene or a material comprising polyolefins and/or polystyrenes are weighed before and/or after said polyolefins and/or polystyrene or the material have been contacted with an enzyme, micro-organism or host cell (or any combination thereof). The presence, absence or level of degradation products of a polyolefin and/or a polystyrene, e.g. degraded by an enzyme, micro-organism or host cell, can be detected or measured by any suitable method known in the art. Non-limiting examples of suitable detection and/or measuring methods include liquid chromatography, gas chromatography, mass spectrometry or any combination thereof (e.g. ESI-MS/MS, MaldiTof, RPHPLC, GC-MS or LC-TOF-MS) of samples, optionally after cultivating a micro-organism or host cell e.g. 1-11 hours, 11-100 hours, or 100 hours-12 months (e.g. one, two, three, four, five, six, seven, eight, nine, ten or 11 months) or even longer in the presence of polyolefins and/or polystyrenes (such as plastics or synthetic polymers) or after allowing a micro-organism, polypeptide or enzyme to contact with polyolefins and/or polystyrene. Other examples of suitable detection and/or measuring methods (including methods of fractionating, isolating or purifying degradation products) include but are not limited to filtration, solvent extraction, centrifugation, affinity chromatography, ion exchange chromatography, electrophoresis, hydrophobic interaction chromatography, gel filtration chromatography, reverse phase chromatography, chromatofocusing, differential solubilization, preparative disc-gel electrophoresis, isoelectric focusing, HPLC, gel permeation chromatography (GPC), fourier-transform infrared spectroscopy (FT-IR), NMR and/or reversed-phase HPLC.

For degradation, polyolefins and/or polystyrenes or a material comprising polyolefins and/or polystyrenes can be contacted with an enzyme, micro-organism or host cell (or any combination thereof) at a ratio, concentration and/or temperature for a time sufficient for the degradation of interest. Suitable time for allowing the enzyme, micro-organism, or host cell to degrade polyolefins and/or polystyrenes can be selected e.g. from the group consisting of 1, 2, 3, 4, 5, 6, 7, 8, 9, 10, 11, 12, 13, 14, 15, 16, 17, 18, 19, 20, 21, 22, 23, and 24 hours, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31 days, 1, 2, 3, 4, 5, 6, 7, 8, 9, 10, 11, and 12 weeks, and 1, 2, 3, 4, 5, 6, 7, 8, 9, 10, 11 and 12 months. The degradation may take place in liquid, semi-solid, moist or dry conditions. The degradation is conveniently conducted aerobically, microaerobically and/or anaerobically. If desired, specific oxygen uptake rate can be used as a process control. The degradation can be conducted continuously, batch-wise, feed batch-wise or as any combination thereof.

In one embodiment the enzyme(s), micro-organism(s) or host cell(s) can be utilized for degrading polyolefins and/or polystyrenes e.g. at a temperature below 100° C. such as 15-95° C., 30-95° C. or 40-80° C. (e.g. 50° C.). In one embodiment the enzyme, fragment, micro-organism or host cell is capable of degrading a polyolefin and/or a polystyrene at a temperature of at least 20° C., at least 25° C., at least 30° C., or at least 37° C. This indicates low energy need and therefore also moderate costs of the method.

In some embodiments of the invention an enzyme and/or enzymes (e.g. a combination of different enzymes) can produce material (e.g. degradation products (such as alkane) or modified material) for other enzymes or enzymes of other type(s) or micro-organisms to further degrade or modify said material (e.g. to fatty acids, PHA or diacids). On the other hand, in some embodiments of the invention a micro-organism, host cell, micro-organisms (e.g. a combination of different micro-organisms) or host cells can produce material (e.g. degradation products (such as alkane) or modified material) for micro-organisms of other type(s) or enzymes to further degrade or modify said material (e.g. to fatty acids, PHA or diacids).

In some embodiments of the present invention the micro-organisms or host cells are cultured under conditions (e.g. suitable conditions) in which the cultured micro-organism or host cell produces polypeptides, enzymes or compounds or interest (e.g. enzymes for degrading polyolefins and/or polystyrenes). The micro-organisms or host cells can be cultivated in a medium containing appropriate carbon sources together with other optional ingredients selected from the group consisting of nitrogen or a source of nitrogen (such as amino acids, proteins, inorganic nitrogen sources such as nitrate, ammonia, urea or ammonium salts), yeast extract, peptone, minerals and vitamins, such as KH2PO4, Na2HPO, MgSO, CaCl2, FeCls, ZnSO, citric acid, MnSO, COCl2, CuSO, Na2MoO4, FeSO4, HsBO4, D-biotin, Ca-Pantothenate, nicotinic acid, myoinositol, thiamine, pyridoxine, p-amino benzoic acid. Suitable cultivation conditions, such as temperature, cell density, selection of nutrients, and the like are within the knowledge of a skilled person and can be selected to provide an economical process with the micro-organism in question. Temperatures may range from above the freezing temperature of the medium to about 50° C. or even higher, although the optimal temperature will depend somewhat on the particular micro-organism. In a specific embodiment the temperature is from about 25 to 35° C. The pH of the cultivation process may or may not be controlled to remain at a constant pH, but is usually between 3 and 9, depending on the production organism. Optimally the pH can be controlled e.g. to a constant pH of 7-8 (e.g. in the case of Escherichia coli) or to a constant pH of 5-6 (e.g. in the case of Yarrowia lipolytica).

Suitable buffering agents include, for example, calcium hydroxide, calcium carbonate, sodium hydroxide, potassium hydroxide, potassium carbonate, hydrogen chloride, sodium carbonate, ammonium carbonate, ammonia, ammonium hydroxide and/or the like. In general, those buffering agents that have been used in conventional cultivation methods are also suitable here. The cultivation conditions can also include oxides, such as ZnO, MnO and/or TiO₂, which may affect positively on the degradation ability of the enzyme.

The micro-organisms or host cells can be normally separated from the culture medium after cultivation, before or after contacting with a polyolefin and/or a polystyrene. The separated micro-organisms, host cells or a liquid (e.g. culture medium) comprising micro-organisms or host cells can be used for contacting polyolefins and/or polystyrenes.

Polypeptides or enzymes can be secreted outside of the cells or they can stay in the cells. Therefore, the polypeptides or enzymes can be recovered from the cells or directly from the culture medium. In some embodiments both intracellular and extracellular polypeptides or enzymes are recovered. Prior to recovering, cells can be disrupted. Isolation and/or purification of polypeptides or enzymes can include one or more of the following: size exclusion, desalting, anion and cation exchange, based on affinity, removal of chemicals using solvents, extraction of the soluble proteinaceous material e.g. by using an alkaline medium (e.g. NaOH, Borate-based buffers or water is commonly used), isoelectric point-based or salt-based precipitation of proteins, centrifugation, and ultrafiltration. In one embodiment of the method, polypeptide or enzyme of the present invention, said polypeptide or enzyme is a purified or partly purified polypeptide or enzyme. If the polypeptide or enzyme is secreted outside of the cell it does not necessarily need to be purified.

In one embodiment, the enzyme or fragment thereof is immobilized. Immobilization can be carried out by any method known to a person skilled in the art such as immobilization by crosslinking e.g. with glutaraldehyde or by using hydrophopic carrier for the enzyme.

Polyolefin(s) and/or polystyrene(s) degrading enzymes can be expressed in any suitable host (cell). Examples of suitable host cells include but are not limited to cells of micro-organisms such as bacteria, yeast, fungi and filamentous fungi, as well as cells of plants and animals (such as mammals). Specific examples of host cells include but are not limited to Escherichia coli, Saccharomyces cerevisiae, Yarrowia lipolytica, Pichia pastoris, Trichoderma reesei, Aspergillus nidulans, Aspergillus niger, Bacillus licheniformis, Bacillus subtilis, and Myceliophthora thermophila.

In one embodiment of the invention the micro-organism(s) or host cell(s) is (are) a bacterium or bacteria or fungus selected from the group comprising or consisting of Bacillus, Paenibacillus, Achromobacter, Acinetobacter, Alcanivorax, Aneurinibacillus, Arthrobacter, Aspergillus, Brevibacillus, Brucella, Chitinophaga, Citrobacter, Comamonas, Cordyceps, Cupriavidus, Delftia, Enterobacter, Escherichia, Exiguobacterium, Flavobacterium, Fusarium, Halomonas, Hyphomicrobium, Klebsiella, Kocuria, Leucobacter, Lysinibacillus, Macrococcus, Methylobacterium, Methylocella, Microbacterium, Micrococcus, Moraxella, Mucor, Nesiotobacter, Nocardia, Ochrobactrum, Pantoea, Paracoccus, Penicillium, Pleurotus, Pseudomonas, Rahnella, Ralstonia, Rhizobium, Rhodococcus, Serratia, Sphingobacterium, Staphylococcus, Stenotrophomonas, Streptococcus, Streptomyces, Trichoderma, Vibrio, Virgibacillus and Xanthobacter; or the enzyme is an enzyme of a bacterium or fungus selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp, Aspergillus awamori, Aspergillus flavus, Aspergillus fumigatus, Aspergillus glaucus, Aspergillus niger, Aspergillus oryzae, Aspergillus sp. Aspergillus sydowii, Aspergillus terreus, Bacillus amyloliquefaciens, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Brevibacillus sp., Brucella anthropi, Chitinophaga sp., Citrobacter amalonaticus, Comamonas sp., Cordyceps confragosa, Cupriavidus necator, Delftia sp., Delftia tsuruhatensis, Enterobacter asburiae, Enterobacter sp., Escherichia coli, Exiguobacterium sp. Flavobacterium sp., Flavobacterium petrolei, Flavobacterium pectinovorum, Flavobacterium aquicola, Fusarium solani, Fusarium sp., Halomonas venusta, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus luteus, Micrococcus lylae, Moraxella sp., Mucor circinelloides, Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Penicillium chrysogenum, Penicillium oxalicum, Penicillium ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Sphingobacterium multivorum, Staphylococcus epidermidis, Staphylococcus cohnii, Staphylococcus sp., Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Vibrio alginolyticus, Vibrio parahaemolyticus, Virgibacillus halodenitrificans, Xanthobacter autotrophicus, and Xanthobacter tagetidis. and any combination thereof.

Also, the micro-organism or host cell of the present invention can be used in a combination with any other micro-organism (simultaneously or consecutively), e.g. micro-organisms can be a population of different micro-organisms degrading different hydrocarbon chains or micro-organisms can be a combination of a bacterium and a fungus (to be used simultaneously or consecutively).

The inventors of the present disclosure have been able to isolate enzymes capable of degrading polyolefins and/or polystyrenes from micro-organisms, and use said enzymes or micro-organisms for degrading polyolefins and/or polystyrenes, and/or producing degradation products of interest.

The present invention further relates to use of the enzyme, micro-organism, host cell, polynucleotide, expression vector or plasmid of the present invention or any combination thereof for degrading a polyolefin and/or a polystyrene or polyolefins and/or polystyrenes of different types.

Also, the present invention concerns a method of producing the enzyme of the present invention, wherein a recombinant micro-organism or host cell comprising the polynucleotide encoding the enzyme or fragment thereof of the present invention expresses or is allowed to express said enzyme or fragment thereof. For example, a vector or plasmid comprising the polynucleotide of interest can be transfected to a host cell, and the host cell can be used for expressing the enzyme of the present invention. In one embodiment the polynucleotide of interest is integrated into the genome of the host cell or the polynucleotide of interest is expressed from a vector or plasmid which is not integrated into the genome of the host cell. In one embodiment said expression of the enzyme can be controlled for example through inducible elements of promoters, vectors or plasmids.

As used in the present disclosure, the terms “polypeptide” and “protein” are used interchangeably to refer to polymers of amino acids of any length. As used herein “an enzyme” refers to a protein or polypeptide which is able to accelerate or catalyze (bio) chemical reactions.

As used herein “polynucleotide” refers to any polynucleotide, such as single or double-stranded DNA (genomic DNA or cDNA or synthetic DNA) or RNA (e.g. mRNA or synthetic RNA), comprising a nucleic acid sequence encoding a polypeptide in question or a conservative sequence variant thereof. Conservative nucleotide sequence variants (i.e. nucleotide sequence modifications, which do not significantly alter biological properties of the encoded polypeptide) include variants arising from the degeneration of the genetic code and from silent mutations.

As used herein “isolated” enzymes, polypeptides or polynucleotides refer to enzymes, polypeptides or polynucleotides purified to a state beyond that in which they exist in cells. Isolated polypeptides, proteins or polynucleotides include e.g. substantially purified (e.g. purified to at least 10%, 20%, 30%, 40%, 50%, 60%, 70%, 80%, 90% purity) or pure enzymes, polypeptides or polynucleotides.

It is well known that a deletion, addition or substitution of one or a few amino acids of an amino acid sequence of an enzyme does not necessarily change the catalytic properties of said enzyme. Therefore, the invention also encompasses variants and fragments of the enzymes of the present invention or given amino acid sequences having the stipulated enzyme activity. The term “variant” as used herein refers to a sequence having minor changes in the amino acid sequence as compared to a given sequence. Such a variant may occur naturally e.g. as an allelic variant within the same strain, species or genus, or it may be generated by mutagenesis or other gene modification. It may comprise amino acid substitutions, deletions or insertions, but it still functions in substantially the same manner as the given enzymes, in particular it retains its catalytic function as an enzyme (e.g. capability to degrade a hydrocarbon chain). In one embodiment of the invention a fragment of the enzyme is an enzymatically active fragment or variant thereof.

A “fragment” of a given enzyme or polypeptide sequence means part of that sequence, e.g. a sequence that has been truncated at the N- and/or C-terminal end. It may for example be the mature part of an enzyme or polypeptide comprising a signal sequence, or it may be only an enzymatically active fragment of the mature enzyme or polypeptide.

It will be obvious to a person skilled in the art that, as the technology advances, the inventive concept can be implemented in various ways. The invention and its embodiments are not limited to the examples described below but may vary within the scope of the claims.

### EXAMPLES

### Example 1. Expression of Bacillus Flexus, Bacillus Licheniformis, Bacillus Subtilis and Bacillus cereus M28 Family Peptidases in Escherichia coli

The gene encoding Bacillus licheniformis M28 family peptidase (SEQ ID NO: 4) amino acid was cloned from genomic Bacillus licheniformis DNA by PCR by using oligonucleotides oPlastBug-124 (ACAATTCCTCTAGAAATAATTTTGTTTAACTTTAAGAAGGAGATATATCCATGA AGAGAAAAATGATGATGTTCGGATTGGC, SEQ ID NO: 11) and oPlastBug-125 (TTGTTAGCAGCCGGATCAAGCTGGGATTTAGGTGACACTATAGAATACTCTT ACTGGGCAACTGAGCTGTAAGACG, SEQ ID NO: 12). The resulting DNA fragment containing coding region of the gene (SEQ ID NO: 1) was cloned into Ncol and Hindlll digested E. coli expression vector pBAT4 with Gibson assembly resulting in plasmid pPB038-4 and expressed E. coli strain SHuffle® T7 Express (NEB, US).

The gene encoding Bacillus flexus M28 family peptidase (SEQ ID NO: 2) amino acid was cloned from genomic Bacillus flexus DNA by PCR by using oligonucleotides oPlastBug-134 (ACAATTCCTCTAGAAATAATTTTGTTTAACTTTAAGAAGGAGATATATCCATGA AAAGAACAAAAAAGACAATTGTATCCGTTGCT, SEQ ID NO: 9) and oPlastBug-135 (TTGTTAGCAGCCGGATCAAGCTGGGATTTAGGTGACACTATAGAATACTCTT ATTTAAGATCTTGCTGATAATAAAGCTCAGGTGC, SEQ ID NO: 10). The resulting DNA fragment containing coding region of the gene (SEQ ID NO: 3) was cloned into Ncol and HindIll digested Escherichia coli expression vector pBAT4 with Gibson assembly resulting in plasmid pPB043-3 and expressed in E. coli strain SHuffle® T7 Express (NEB, US).

The gene encoding Bacillus subtilis M28 family peptidase (SEQ ID NO: 6) amino acid sequence was commercially (GenScript, US) synthetized with codon optimization for expression in E. coli cells (SEQ ID NO: 5). The insert bearing the coding gene was cleaved from pUC57 vector with FastDigest™ Xbal and Hindlll (Thermo Fisher Scientific, US) enzymes. The fragment was ligated with T4 DNA ligase (NEB, US) into pBAT4 vector previously digested with the same enzymes and transformed into E. coli TOP10 competent cells (Thermo Fisher Scientific, US). The resulting plasmid pPB121-1 was then transformed and expressed in E. coli SHuffle® T7 Express cells (NEB, US).

The gene encoding Bacillus cereus M28 family peptidase (SEQ ID NO: 8) amino acid sequence was commercially (GenScript, US) synthetized with codon optimization for expression in E. coli cells (SEQ ID NO: 7). The insert bearing the coding gene was cleaved from pUC57 vector with FastDigest™ Xbal and Hindlll (Thermo Fisher Scientific, US) enzymes. The fragment was ligated with T4 DNA ligase (NEB, US) into pBAT4 vector previously digested with the same enzymes and transformed into E. coli TOP10 competent cells (Thermo Fisher Scientific, US). The resulting plasmid pPB129-1 was then transformed and expressed in E. coli SHuffle® T7 Express cells (NEB, US).

Plasmids pPB038-4, pPB121-1 and pPB129-1 were expressed in E. coli SHuffle® T7 Express cells grown on 50 mL of Super Broth medium (30 g·L⁻¹ tryptone, 20 g·L⁻¹ yeast extract, 10 g·L⁻¹ MOPS (3-[N-morpholino]-propanesulfonic acid) supplemented with 100 μg·mL⁻¹ ampicillin at 30° C. and 220 rpm. Plasmid pPB043-3 was expressed in the same competent E. coli cells grown on 50 ml of Terrific Broth medium (13.3 g·L⁻¹ tryptone, 26.6 g·L⁻¹ yeast extract, 5.5 g·L⁻¹ glycerol supplemented with 100 μg·mL⁻¹ ampicillin and a 10× salt solution (23.1 g·L⁻¹ KH2PO4 and 125.4 g·L⁻¹ K2HPO4)) at 30° C. and 220 rpm. Protein expression was induced by the addition of β-D-1-thiogalactopyranoside (IPTG) at the final concentration of 1 mM when the cultures achieved OD600 of 0.6-0.8. Induced cultures were then incubated at 30° C. for 16-20 h. Cells were harvested by centrifugation (4,000 rpm for 10 min at room temperature), and extracellular supernatant was collected and filtered through 0.20 μm filter. Extracellular supernatant was used directly in enzyme assays or was partially purified. Recombinant enzymes from filtered samples were partially purified as follows: pH was adjusted to pH 8.5 with 1 M HCl and samples were diluted with 1 M Tris-HCl pH 8.5 to the final concentration of 50 mM. Next, 2 ml of samples were loaded into HiTrap® Q HP column (1 ml column volume, Cytiva, US) equilibrated with 50 mM Tris-HCl pH 8.5 followed by washing steps with 5 ml of 50 mM Tris-HCl pH 8.5. Enzymes were eluted with 2 ml of 0.25 M NaCl, 50 mM TrisHCl, pH 8.5. Purity of enzymes were detected with SDS-PAGE analysis. Partially purified enzymes were used directly in enzyme assays. As a negative control purification steps were repeated with E. coli strain expressing empty pBAT4 plasmid.

### Example 2. Degradation of Polypropylene, Polyethylene and Polystyrene with Bacillus licheniformis Enzyme

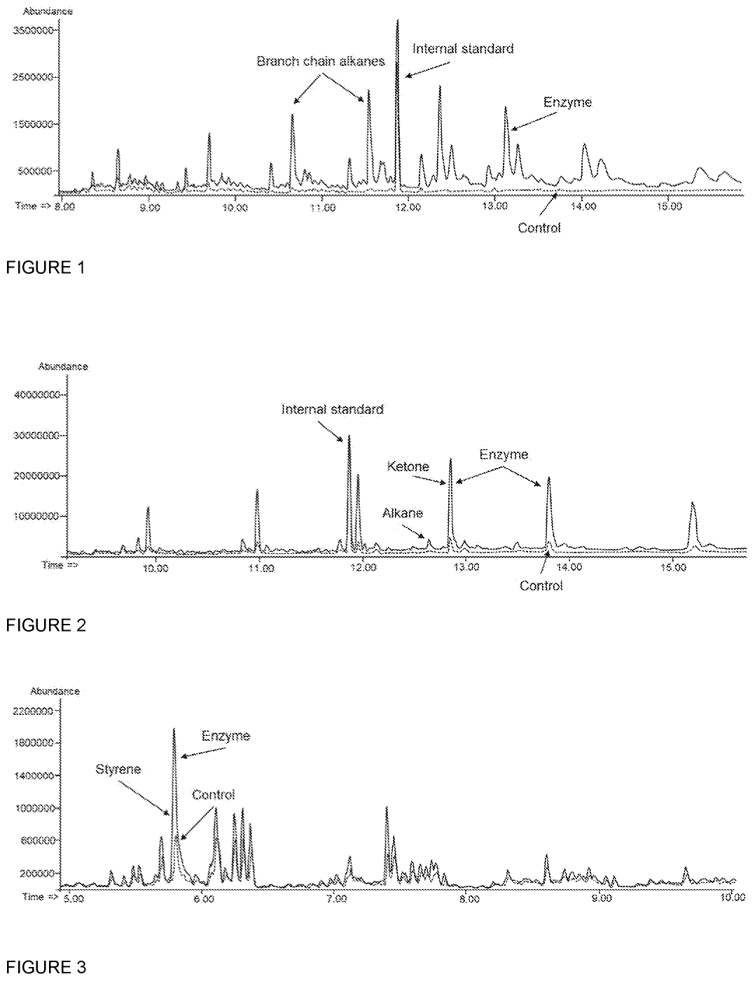

Enzyme assay with polypropylene with Bacillus licheniformis enzyme was carried out with partially purified enzyme as follows: 950 ul of 50 mM HEPES buffer (pH 8.0) and 10 mg of polypropylene powder (Licocene pp 6102 Fine grain, Clariant) were incubated with 50 μl of partially purified enzymes from Example 1 at 50° C. for 72 h. 0.25 M NaCl, 50 mM Tris-HCl PH 8.5 elution sample from the purification of E. coli strain having empty plasmid (pBAT4) as described in Example 1 was used as a control. After incubation GC-MS run was carried out with liquid fraction as described in Example 3. Results from the GC-MS run are shown in FIG. 1. With enzyme samples several peaks appeared which were missing from control sample. These peaks presented alkane like compounds or oxygen containing hydrocarbons.

Enzyme assay with polyethylene with Bacillus licheniformis enzyme was carried out with partially purified enzyme as follows: 950 μL of 50 mM HEPES buffer (pH 8.0) and 10 mg of polyethylene powder (Mw 4,000 Da, Sigma-Aldrich, US) were incubated with 50 μL of partially purified enzymes from Example 1 at 50° C. for 72 h. 0.25 M NaCl, 50 mM Tris-HCl pH 8.5 elution sample from the purification of E. coli strain having empty plasmid (pBAT4) as described in Example 1 was used as a control. After incubation GC-MS run was carried out with liquid fraction as described in Example 3. Results from the GC-MS run are shown in FIG. 2. With enzyme samples several peaks appeared which were missing from control sample. These peaks presented alkane like compounds or oxygen containing hydrocarbons.

Enzyme assay with polystyrene with Bacillus licheniformis enzyme was carried out with partially purified enzyme as follows: 950 μL of 50 mM HEPES buffer (pH 8.0) and 10 mg of polystyrene powder (particle size 900 μm, density 0.62 g/mL, GoodFellows Cambridge Ltd., England) were incubated with 50 μL of partially purified enzymes from Example 1 at 50° C. for 168 h at 200 rpm. 0.25 M NaCl, 50 mM TrisHCl pH 8.5 elution sample from the purification of E. coli strain having empty plasmid (pBAT4) as described in Example 1 was used as a control. After incubation GC-MS run was carried out with liquid fraction as described in Example 3. Results from the GC-MS run are shown in FIG. 3. With enzyme samples several peaks appeared which were missing from control sample. These peaks presented alkane like compounds or oxygen containing hydrocarbons.

### Example 3. Gas Chromatography-Mass Spectrometry (GC-MS) Analysis of Volatile Degradation Products of Hydrocarbons with Peptidase

Aliquots (300 μL) of the samples from Example 2 were transferred to new tubes and an internal standard (methyl heptadecanoate) was added. The samples were extracted with dichloromethane (200 μL) by agitating in a shaker for 15 min. After extraction the samples were allowed to settle (15 min at room temperature), centrifuged (5 min, 10,000 rpm) and finally, the dichloromethane phase was transferred to GC-MS vials. The runs were performed on Agilent GC-MS equipped with an HPFFAP (25 m×200 μm×0.3 μm) column and helium was used as a carrier gas. The injector temperature was 250° C., and a splitless injection mode was set. The oven temperature was 40° C. for 3 min, increased to 240° C. at 20° C./min and kept at 240° C. for 14 min. The detected mass range was 35-600 m/z and compounds were identified based on NIST08 MS library. Results from GC-MS analysis are described in Examples 2 and 6.

### Example 4. FTIR Analysis of Polypropylene Sample Incubated with B. Licheniformis Peptidase

Enzyme assay with PP powder with Bacillus licheniformis enzyme was carried out with partially purified enzyme as it follows. Approximately 10 μg of the partially purified enzyme from Example 1 were incubated with 30 mg of PP powder (Mw 12,000 Da, average Mn 5,000 Da, density 0.9 g/mL, Sigma-Aldrich, US) and diluted with 50 mM HEPES buffer pH 8.0 in a final volume of 1 mL. The enzyme reaction was performed at 50° C. for 168 h in glass vials. The same amount of the partially purified enzyme was denatured at 98° C. for 10 min and used as a negative control. FTIR was carried out with Nicolet™ IS50 FT-IR spectrophotometer (Thermo Fisher Scientific, US) with following parameters: diamond crystal, resolution of 4 cm⁻¹, 32 scans, log (1/R) final format, peak-to-peak 2.53, loc 2048, with gain 1. Areas of selected peaks were analysed and compared to the areas of peaks 2949-2837 cm⁻¹ which correspond to C—H groups. In comparison higher peak areas could be seen for the peaks at 3406, 1650 and 1043 cm⁻¹ which correspond to O—H, C═C and C—O bonds, respectively (Table 1). These results indicate that B. licheniformis peptidase modifies PP polymer by e.g. adding hydroxy groups, oxygen and generating double bonds.

TABLE 1
Comparison of FT-IR peak areas of PP powder incubated
with B. licheniformis peptidase and the control. Values
are relative values compared to peaks 2949-2837.

| | |
|---|---|
| Peak wavenumber | Areas |

| (cm⁻¹) | Assignment | Enzyme | Control |
|---|---|---|---|
| 3406 | O—H | 0.97 | 0.14 |
| 2949-2837 | C—H | 1.00 | 1.00 |
| 1650 | C═C | 0.10 | 0.03 |
| 1454 | C—H | 0.17 | 0.18 |
| 1043 | C—O | 0.14 | 0.02 |
| 998 | C—C | 0.04 | 0.03 |
| 595 | O—H | 0.02 | 0.00 |

### Example 5. Size Exclusion Chromatography (SEC) Analysis of Pre-Treated Polystyrene Film Incubated with B. licheniformis Peptidase and Toluene

Enzyme assay with pre-treated polystyrene film with Bacillus licheniformis enzyme was carried out with filtered enzyme from Example 1 as follows: 2 cm×2 cm PS film previously washed with ethanol 70% (v/v) was added in a beaker with 30 mL of 10 mM ZnO. PS film was treated with microwave at 900 W for 2 min. Pre-treated PS film was added in the reaction containing 50 μg of enzyme sample from Example 1 diluted in 500 μL of 50 mM HEPES (pH 8.0) and 500 μL of toluene. A reaction without enzyme was used as a control. Samples (triplicates) were incubated at 30° C. with agitation of 200 rpm for 162 h. After incubation HEPES buffer fraction was removed and toluene fraction containing dissolved PS film was evaporated with a continuous stream of nitrogen gas. After evaporation SEC analysis was carried out as it follows: the PS samples were dissolved overnight using chloroform (concentration of 3 mg/mL). In all cases, the samples were filtered (0.45 μm) before the measurement. The SEC measurements were performed in chloroform eluent (0.6 mL/min, 30° C.) using Styragel HR 4 and 3 columns (Waters Co., US) with a pre-column. The elution curves were detected using 2414 Refractive index detector (Waters Co., US). The molar mass distributions (MMD) were calculated against nine PS standards (1,260-3,040,000 g/mol), using Empower 3 software (Waters Co., US). Results from size exclusion chromatography (SEC) analysis are showed in Table 2.

TABLE 2
Results of size exclusion chromatography (SEC) analysis.
Results are average from triplicate samples

| | Mn¹ | Mw² | PDI³ |
|---|---|---|---|
| Enzyme | 157947 | 387901 | 2.5 |
| Control | 186547 | 405088 | 2.2 |
| Change % | 15.3 | 4.2 | 13.6 |

¹Mn: number average molecular weight;
²Mw: weight average molecular weight;
³PDI: polydispersity (Mw/Mn).

In triplicate enzyme samples Mn and Mw of PS film were decreased about 15 and 4%, respectively, and polydispersity was increased about 14%. These results indicate that peptidase can degrade PS in the presence of toluene.

### Example 6. Classification and Identification of Functional Domains from Polyethylene, Polypropylene and Polystyrene Degrading Enzymes

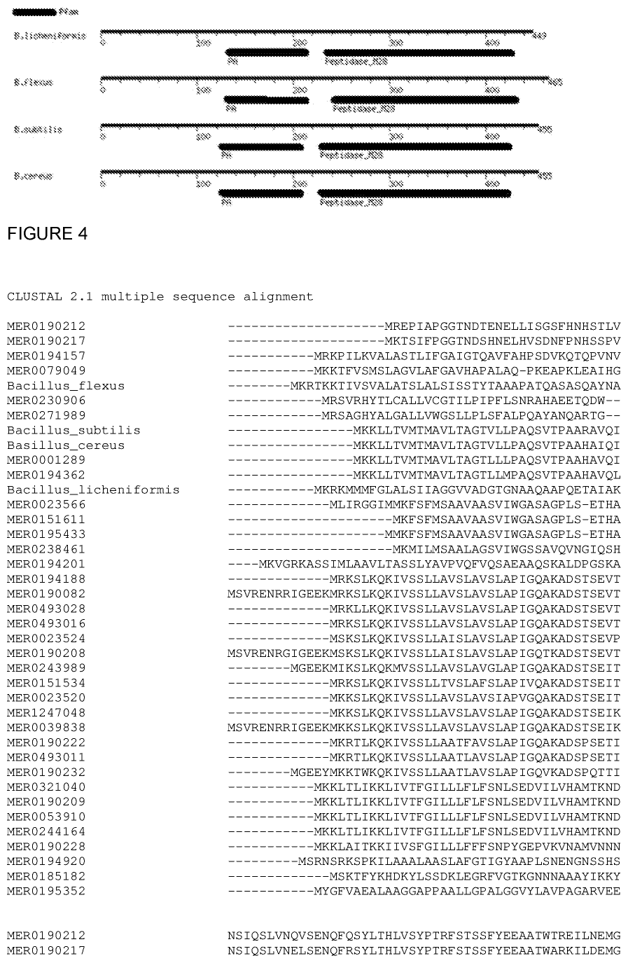

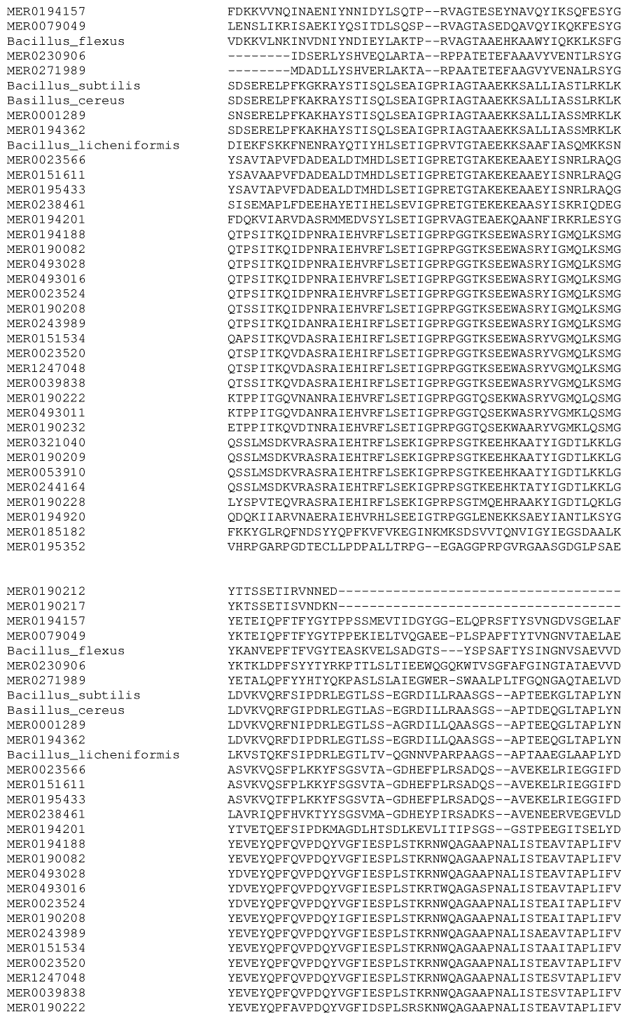

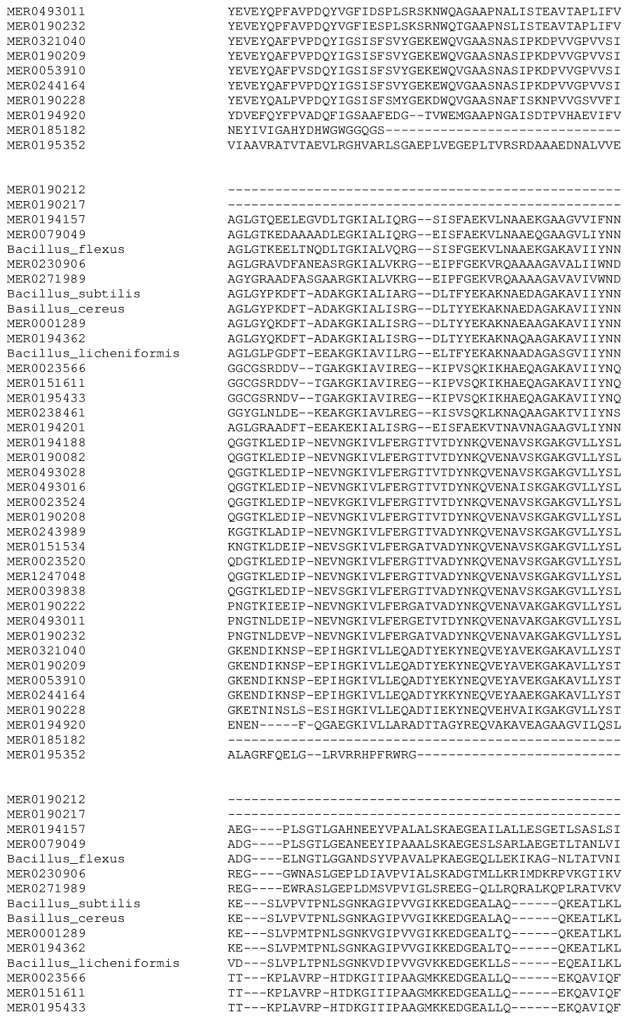

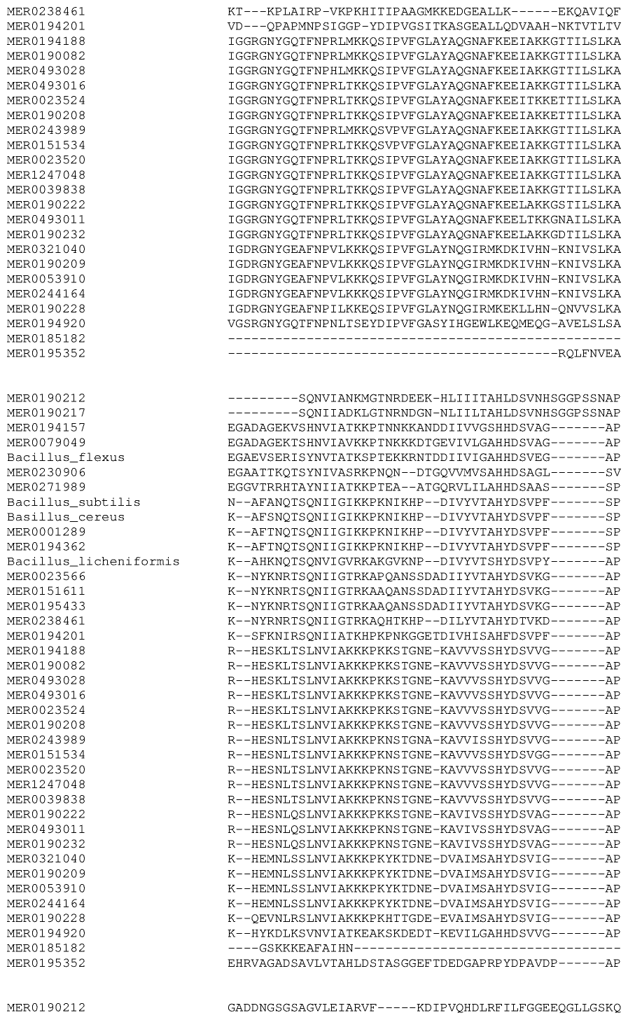

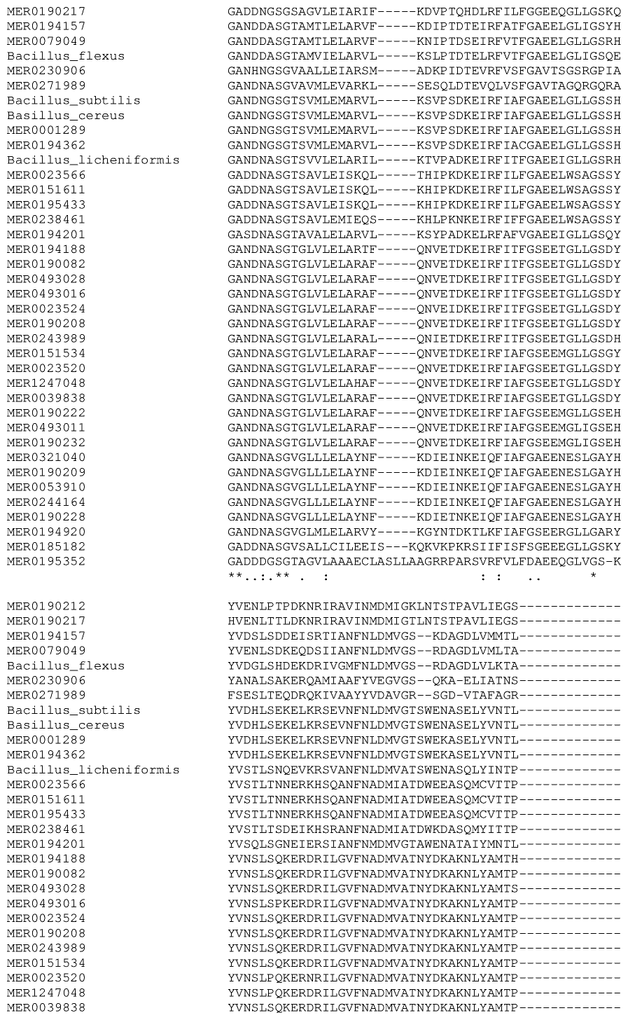

The four M28 family peptidases from B. licheniformis (SEQ ID NO: 4), B. flexus (SEQ ID NO: 2), B. subtilis (SEQ ID NO: 6) and B. cereus (SEQ ID NO: 8) were classified according to their catalytic type, family and subfamily from the Peptidase database MEROPS (https://www.ebi.ac.uk/MEROPS/). Functional domains were also verified according to Pfam (http://pfam.xfam.org/), InterPro (http://www.ebi.ac.uk/interpro/) and UniProt (https://www.uniprot.org/) databases. The M28 family peptidases from B. licheniformis (SEQ ID NO: 4), B. flexus (SEQ ID NO: 2), B. subtilis (SEQ ID NO: 6) and B. cereus (SEQ ID NO: 8) were all identified as enzymes from subfamily M28F of M28 family peptidases which contains aminopeptidases and carboxypeptidases. They belong to clan MH of metalloproteases and include YwaD peptidases (MEROPS-ID M28.009), AM-1 aminopeptidases (MEROPS-ID M28.020), unassigned peptidases (MEROPS-ID M28.UPF) and nonpeptidase homologues (MEROPS-ID M28.UNF). Functional annotation revealed that the four peptidases have protease-associated (PF02225) and/or peptidase family M28 (PF04389) domains predicted by Pfam (FIG. 4). And according to InterPro, the following domains can be found in their sequences: protease associated domain (IPR003137), peptidase M28 (IPR007484), glutamate carboxypeptidase 2-like (IPR039373) and aminopeptidase Y (IPR029514).

### Example 7. Characterisation of Conservative Amino Acid Sequence Motifs of Polyethylene, Polypropylene and Polystyrene Degrading M28 Family Peptidases

Amino acid sequences originating from species which have been shown to degrade polyethylene, polypropylene or polystyrene were collected from Peptidase database MEROPS (https://www.ebi.ac.uk/MEROPS: MEROPS:/) belonging to M28F subfamily. These collected sequences (SEQ ID Nos: 2, 4, 6, 8, 13-47) were used in multiple sequence alignment carried out with CLUSTAW (https://www.genome.jp/tools-bin/clustalw) with default parameters.

In the alignment several consensus amino acids could be detected (based on Bacillus licheniformis amino acid position): Gly264, Ala265, Ser270, Gly271 and Gly305 (see FIG. 5). Additionally, some of the enzymes were lacking protease associated domain identified in examples 6 and seen in 3D structure of Bacillus licheniformis peptidase in Example 8.

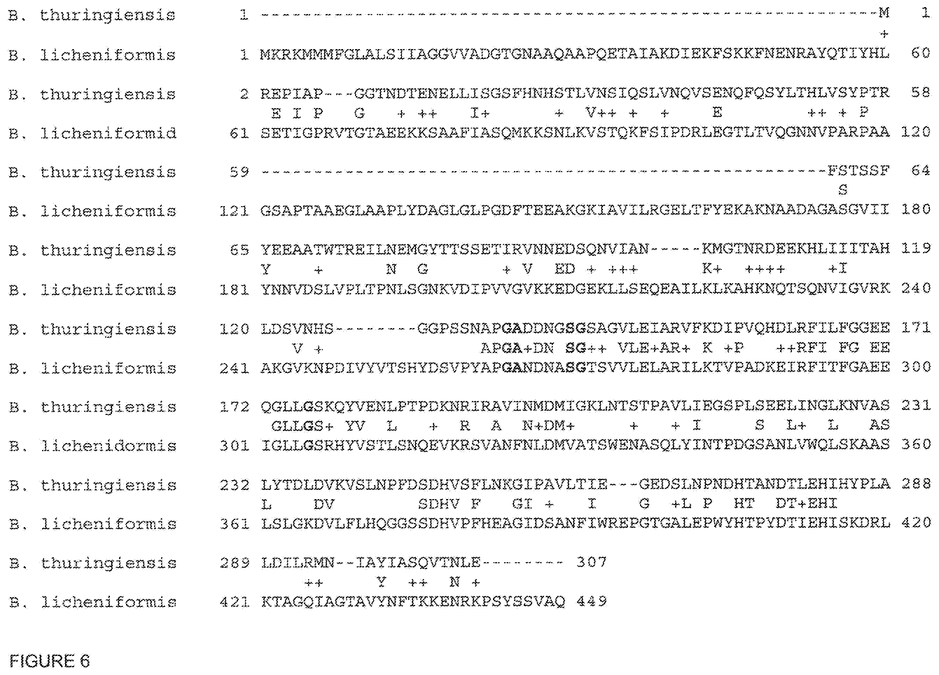

To confirm the existence and position of consensus amino acids in a specific enzyme corresponding amino acid sequence was compared to B. licheniformis M28 family peptidase (SEQ ID NO: 4) by carrying out pairwise alignment with ClustalW default parameters by using Geneious 10.2.6 software. In FIG. 6 there is an example of pairwise alignment between Bacillus licheniformis M28 family peptidase (SEQ ID NO: 4) and B. thuringiensis M28 family peptidase (SEQ ID NO: 39). Even these amino acid sequences have only 18% identity between each other above-mentioned consensus amino acids could be detected and their position in Bacillus licheniformis M28 family peptidase amino acid sequence identified (mark in bold).

### Example 8. Localising Consensus Amino Acids into Enzymes 2D and 3D Structure

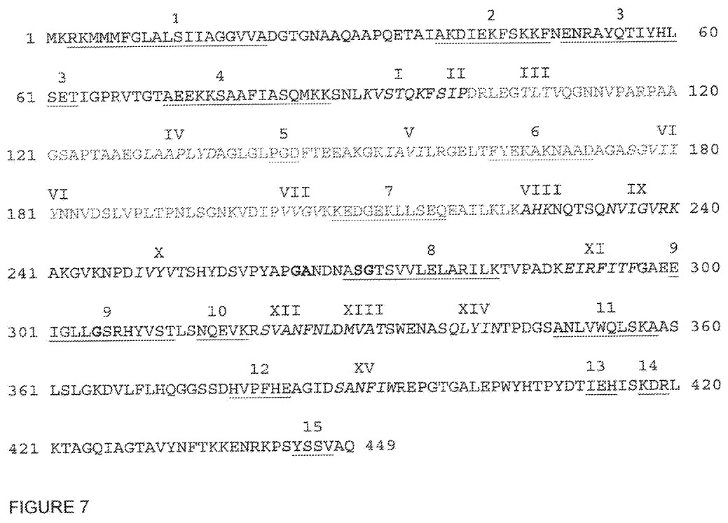

Two-dimensional and 3 D structures of Bacillus licheniformis M28 family peptidase (SEQ ID NO: 4) was constructed with Phyre2 protein homology/analogy recognition engine V 2.0 (http://www.sbg.bio.ic.ac.uk/phyre2/html/page.cgi?id=index) with default parameters. The predicted alfa helixes and beta sheets were localised together with identified consensus amino acids from Example 7 into amino acid sequence shown in FIG. 7. The consensus amino acids from Example 7 were identified in predicted 3D structure. These amino acids were in alfa helixes 8 and 9 (Ser270, Gly271 and G305) or in loop area (Gly264 and Ala265). Three-dimensional structure indicated that enzyme consists of two domains. The smaller protease associated domain is indicated in 2D structure in grey in FIG. 7.

### Example 9. Truncated Bacillus licheniformis M28 Family Peptidase can Degrade Polystyrene

Truncated B. licheniformis M28 family peptidase (SEQ ID NO: 49) was cloned as follows: pBAT plasmid containing 5′ and 3′ prime ends of M28 family peptidase was PCR amplified with oligonucleotides oPlastBug-361 (GTCAAATCTGAAAGTAAGCACACAAAAGTTCAGCATACCTGCTCATAAAAATC AAACCTCGCAAAACGTAATCG, SEQ ID NO: 50) and oPlastBug-362 (GGACGCCGATTACGTTTTGCGAGGTTTGATTTTTATGAGCAGGTATGCTGAA CTTTTGTGTGCTTACTTTCA, SEQ ID NO: 51) by using plasmid pPB038-4 as a template. PCR fragment containing nucleotide sequence of truncated M28 family peptidase (SEQ ID NO: 48) and pBAT4 plasmid was self-ligated with Gibson assembly resulting in plasmid pPB165-2. Truncated peptidase was expressed in E. coli and purified as described in Example 1. The partially purified enzyme was incubated with ZnO-treated PS film in the same conditions as described in Example 5. Liquid fractions were removed, and solid phase was analysed by SEC as aforementioned in Example 5 and with FTIR as described in Example 4. SEC analysis revealed a decrease of 6% in the Mn of PS films and an increase of 9.5% in polydispersity with truncated peptidase from B. licheniformis compared to the denatured enzyme (Table 3). This result indicates that truncated M28 family peptidase can degrade polystyrene.

TABLE 3
SEC analysis of ZnO-treated PS film incubated with truncated
peptidase from B. licheniformis and the control.

| | Mn¹ | PDI² |
|---|---|---|
| Enzyme | 172944 | 2.3 |
| Control | 184151 | 2.1 |
| Change % | 6 | 9.5% |

¹Mn: number average molecular weight;
²PDI: polydispersity (Mw/Mn).

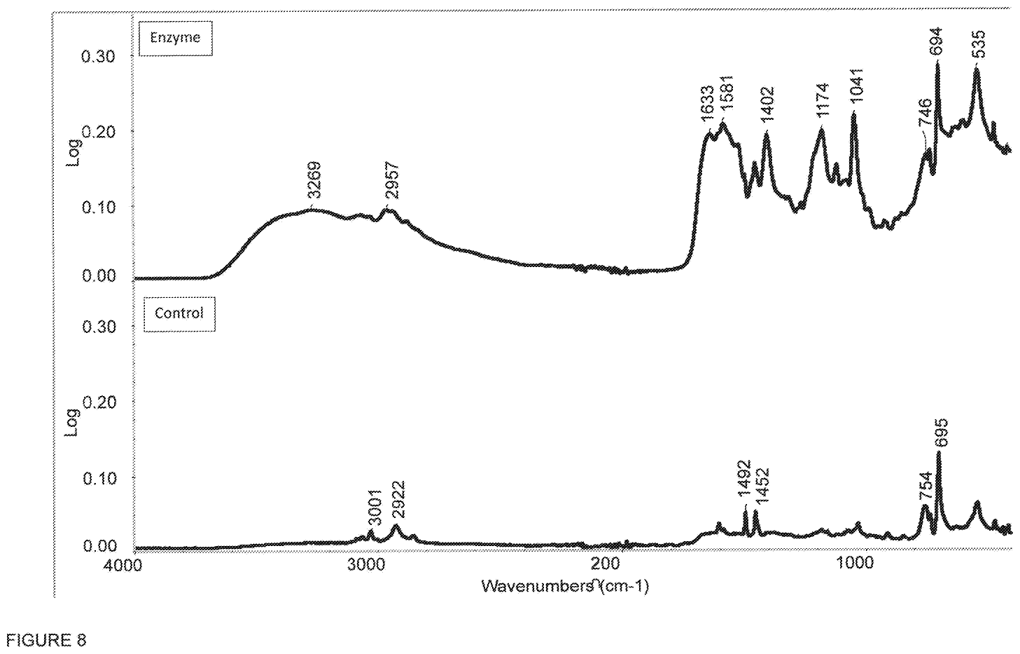

In FTIR analysis several peaks could be detected in the enzyme sample compared to control (FIG. 8). The detected peaks at 3269 and 1043 cm⁻¹ correspond to OH and C—O bonds, respectively, indicates that truncated peptidase can add oxygen to the polystyrene in degradation process.

### Example 10. Expression of Bacillus licheniformis M28 Family Peptidase in Yarrowia lipolytica

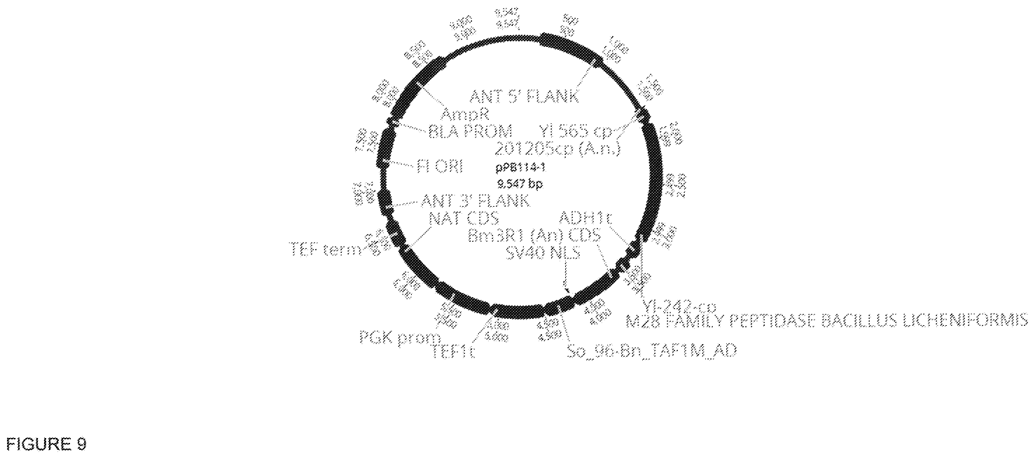

The gene encoding Bacillus licheniformis mature M28 family peptidase (SEQ ID NO: 4) amino acid with Yarrowia lipolytica LIP2 signal peptide (SEQ ID NO: 52) was commercially (Genscript) synthetized with codon optimization for expression in Yarrowia lipolytica cells (SEQ ID NO: 53). Pacl and Bg/ll restriction sites were included at 5′ and 3′ ends of construct for restriction digestion cloning. The constructs were cloned into Y. lipolytica integration cassette plasmid B11157 digested with Pacl and Bc/l. B11157 plasmid contains flanks to ANT1 gene and SES promoter. The resulting plasmid was named as pPB114-1 (FIG. 9). Notl digested integration fragment was transformed into VTT-C-00365 Yarrowia lipolytica strain (VTTCC) with Frozen-EZ yeast Transformation II™ kit (Zymo Research, US). The transformant having B. licheniformis M28 family peptidase integrated was used in the enzyme assay.

The gene encoding truncated version from Bacillus licheniformis M28 family peptidase (SEQ ID NO 49) amino acid with Yarrowia lipolytica LIP2 signal peptide (SEQ ID NO: 54) was PCR amplified with oligos oPlastBug-363 (SEQ ID NO: 56, GTCTAACCTGAAGGTCTCGACCCAGAAGTTCTCGATCCCTGCTCACAAGAAC CAGACCTCTCAGAAC) and oPlastBug-364 (SEQ ID NO:57, GCACTCCGATCACGTTCTGAGAGGTCTGGTTCTTGTGAGCAGGGATCGAGAA CTTCTGGGTCGAG) using plasmid pPB114-1 as a template. PCR fragment containing nucleotide sequence of truncated M28 family peptidase (SEQ ID NO: 55) and B11157 plasmid was self-ligated with Gibson assembly resulting in plasmid pPB166-2. Notl digested integration fragment was transformed into VTT-C-00365 Yarrowia lipolytica strain (VTTCC) with Frozen-EZ yeast Transformation II™ kit (Zymo Research, US). The transformant having truncated B. licheniformis M28 family peptidase integrated was used in the enzyme assay.

After transformation single colonies were cultivated in 3 mL of YPD medium (20 g Bacto peptone, 10 g yeast extract, 20 g glucose per litre) in 24-wells plate. After 1 day incubation at 28° C. with 200 rpm shaking cultures were centrifuged (3184 g, 10 min at RT) and supernatant samples were collected.

The supernatant sample of full (pPB114-1) and truncated (pPB166-2) peptidase from B. licheniformis was also used in the PS powder degradation assay. Briefly, 100 μL of enzyme sample were diluted in 900 μL of 50 mM HEPES buffer (pH 8.0) and incubated with 50 mg of PS powder (info) at 30° C. for 5 days under agitation of 220 rpm in glass vials. The enzyme samples were denaturated at 95° C. for 30 min and used as negative control. The supernatant was removed and the solid phase containing the remaining PS powder was analysed by SEC as described in the Example 5. Both full and truncated peptidases decreased the weight distribution of PS powder (Table 4). Full peptidase (pPB114-1) reduced in 3.6% the Mn of PS powder compared to the control, while the truncated version (pPB166-2) decreased the Mn and Mw in 3.5 and 5.3%, respectively. These results indicate that full length and truncated M28 family peptidase are expressed in active form in Yarrowia lipolytica.

TABLE 4
SEC analysis of PS powder incubated with full and
truncated peptidase from B. licheniformis.

| | B. licheniformis peptidase | | | |
|---|---|---|---|---|
| | Full version (pPB114-1) | | Truncated (pPB166-2) | |
| | Mn¹ | Mw² | Mn¹ | Mw² |
| Enzyme | 92040 | 247562 | 91004 | 241376 |
| Control | 95472 | 249748 | 94317 | 255001 |
| Change (%) | −3.6 | −0.9 | −3.5 | −5.3 |

¹Mn: number average molecular weight;
²Mw: weight average molecular weight.

### Example 11. SEC Analysis Reveals PS Degradation by Peptidases from B. Subtilis and B. Cereus

Peptidases from B. subtilis (pPB121-1) and B. cereus (pPB129-1) were expressed as aforementioned in the Example 1. The E. coli extracellular fraction containing each peptidase was harvested and used in the PS degradation assay. 200 μL of enzyme sample were diluted in 800 μL of 50 mM HEPES buffer (pH 8.0) and incubated with 10 mg of PS powder (info) at 30° C. for 5 days under agitation of 700 rpm in an orbital shaker. MilliQ water was used instead of enzyme sample for the negative control and the reactions were carried out in 96-deep wells plate. The total volume of 1.0 mL was then transferred to glass vials, the supernatant was removed and the solid phase containing the remaining PS powder was analysed by SEC as described in the Example 5. Both peptidases decreased the weight distribution of PS powder (Table 5). Peptidase from B. subtilis (pPB121-1) reduced in 2.8% the Mw of PS powder compared to the control, while B. cereus peptidase (pPB129-1) decreased the Mn and Mw in 2.6 and 1.5%, respectively.

TABLE 5
SEC analysis of PS powder incubated with B. subtilis
(pPB121-1) and B. cereus (pPB129-1) peptidases.

| | B. subtilis | | B. cereus | |
|---|---|---|---|---|
| | peptidase (pPB121-1) | | peptidase (pPB129-1) | |
| | Mn¹ | Mw² | Mn¹ | Mw² |
| Enzyme | 94288 | 250135 | 92433 | 253446 |
| Control | 94921 | 257236 | 94921 | 257236 |
| Change (%) | −0.7 | −2.8 | −2.6 | −1.5 |

¹Mn: number average molecular weight;
²Mw: weight average molecular weight.

1. A method of degrading a polyolefin and/or a polystyrene, said method comprising
providing a material comprising a polyolefin and/or a polystyrene and an enzyme or a fragment thereof capable of degrading the polyolefin and/or the polystyrene, and
allowing said enzyme or fragment thereof to degrade the polyolefin and/or the polystyrene,
wherein the enzyme or fragment thereof belongs to M28F peptidase subfamily, and comprises the amino acids Gly264, Ala265, Ser270, Gly271 and Gly305 corresponding to the amino acid positions presented in SEQ ID NO: 4, and wherein the enzyme or fragment thereof is optionally genetically modified.

2. An isolated enzyme or a fragment thereof belonging to M28F peptidase subfamily and comprises the amino acids Gly264, Ala265, Ser270, Gly271 and Gly305 corresponding to the amino acid positions presented in SEQ ID NO: 4, and wherein said enzyme or fragment is capable of degrading a polyolefin and/or a polystyrene, wherein the enzyme or fragment thereof is optionally genetically modified.

3. A micro-organism or a host cell comprising an enzyme or a fragment thereof belonging to M28F peptidase subfamily and comprises the amino acids Gly264, Ala265, Ser270, Gly271 and Gly305 corresponding to the amino acid positions presented in SEQ ID NO: 4, and wherein said enzyme or fragment is capable of degrading a polyolefin and/or a polystyrene, wherein the enzyme or fragment thereof is optionally genetically modified.

4. (canceled)

5. (canceled)

6. The method of claim 1, wherein the enzyme or fragment thereof has at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, and the enzyme optionally originates from or is an enzyme of a bacterium or fungus selected from the group comprising or consisting of Bacillus, Paenibacillus, Achromobacter, Acinetobacter, Alcanivorax, Aneurinibacillus, Arthrobacter, Aspergillus, Brevibacillus, Brucella, Chitinophaga, Citrobacter, Comamonas, Cordyceps, Cupriavidus, Delftia, Enterobacter, Escherichia, Exiguobacterium, Flavobacterium, Fusarium, Halomonas, Hyphomicrobium, Klebsiella, Kocuria, Leucobacter, Lysinibacillus, Macrococcus, Methylobacterium, Methylocella, Microbacterium, Micrococcus, Moraxella, Mucor, Nesiotobacter, Nocardia, Ochrobactrum, Pantoea, Paracoccus, Penicillium, Pleurotus, Pseudomonas, Rahnella, Ralstonia, Rhizobium, Rhodococcus, Serratia, Sphingobacterium, Staphylococcus, Stenotrophomonas, Streptococcus, Streptomyces, Trichoderma, Vibrio, Virgibacillus and Xanthobacter; or the enzyme is an enzyme of a bacterium or fungus selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp. Aspergillus awamori, Aspergillus flavus, Aspergillus fumigatus, Aspergillus glaucus, Aspergillus niger, Aspergillus oryzae, Aspergillus sp. cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Brevibacillus sp., Brucella anthropi, Chitinophaga sp. Citrobacter amalonaticus, Comamonas sp., Cordyceps confragosa, Cupriavidus necator, Delftia sp., Delftia tsuruhatensis, Enterobacter asburiae, Enterobacter sp., Escherichia coli, Exiquobacterium sp. Flavobacterium sp., Flavobacterium petrolei, Flavobacterium pectinovorum, Flavobacterium aquicola, Fusarium solani, Fusarium sp., Halomonas venusta, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus luteus, Micrococcus lylae, Moraxella sp., Mucor circinelloides, Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Penicillium chrysogenum, Penicillium oxalicum, Penicillium ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Sphingobacterium multivorum, Staphylococcus epidermidis, Staphylococcus cohnii, Staphylococcus sp., Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Vibrio alginolyticus, Vibrio parahaemolyticus, Virgibacillus halodenitrificans, Xanthobacter autotrophicus, and Xanthobacter tagetidis.

7. The isolated enzyme or fragment thereof of claim 2, wherein the enzyme or fragment thereof has at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, and wherein the enzyme optionally originates from or is an enzyme of a bacterium or fungus selected from the group comprising or consisting of Bacillus, Paenibacillus, Achromobacter, Acinetobacter, Alcanivorax, Aneurinibacillus, Arthrobacter, Aspergillus, Brevibacillus, Brucella, Chitinophaga, Citrobacter, Comamonas, Cordyceps, Cupriavidus, Delftia, Enterobacter, Escherichia, Exiguobacterium, Flavobacterium, Fusarium, Halomonas, Hyphomicrobium, Klebsiella, Kocuria, Leucobacter, Lysinibacillus, Macrococcus, Methylobacterium, Methylocella, Microbacterium, Micrococcus, Moraxella, Mucor, Nesiotobacter, Nocardia, Ochrobactrum, Pantoea, Paracoccus, Penicillium, Pleurotus, Pseudomonas, Rahnella, Ralstonia, Rhizobium, Rhodococcus, Serratia, Sphingobacterium, Staphylococcus, Stenotrophomonas, Streptococcus, Streptomyces, Trichoderma, Vibrio, Virgibacillus and Xanthobacter; or the enzyme is an enzyme of a bacterium or fungus selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp, Aspergillus awamori, Aspergillus flavus, Aspergillus fumigatus, Aspergillus glaucus, Aspergillus niger, Aspergillus oryzae, Aspergillus sp. Aspergillus sydowii, Aspergillus terreus, Bacillus amyloliquefaciens, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Brevibacillus sp., Brucella anthropi, Chitinophaga sp., Citrobacter amalonaticus, Comamonas sp., Cordyceps confragosa, Cupriavidus necator, Delftia sp., Delftia tsuruhatensis, Enterobacter asburiae, Enterobacter sp., Escherichia coli, Exiguobacterium sp. Flavobacterium sp., Flavobacterium petrolei, Flavobacterium pectinovorum, Flavobacterium aquicola, Fusarium solani, Fusarium sp., Halomonas venusta, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus luteus, Micrococcus lylae, Moraxella sp., Mucor circinelloides, Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Penicillium chrysogenum, Penicillium oxalicum, Penicillium ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Sphingobacterium multivorum, Staphylococcus epidermidis, Staphylococcus cohnii, Staphylococcus sp., Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Vibrio alginolyticus, Vibrio parahaemolyticus, Virgibacillus halodenitrificans, Xanthobacter autotrophicus, and Xanthobacter tagetidis.

8. The method of claim 1, wherein the polyolefin is polyethylene (PE), cross-linked polyethylene (PEX or XLPE), ultra-high molecular weight polyethylene (UHMWPE), highdensity polyethylene (HDPE), medium-density polyethylene (MDPE), linear low-density polyethylene (LLDPE), low density polyethylene (LDPE), very low density polyethylene (VLDPE), polypropylene (PP), polymethylpentene (PMP), polybutene-1 (PB-1), polyisobutylene (PIB), or any combination thereof.

9. The isolated enzyme or fragment thereof of claim 2, wherein the isolated enzyme or fragment thereof is capable of degrading the polyolefin and/or polystyrene at a temperature of at least 20° C., at least 25° C., at least 30° C., at least 37° C. or at a temperature of 30-95° C.

10. The method of claim 1, wherein at least one or more degradation products selected from the group consisting of an alkane, alkene, alkyne, cycloalkane, alkadiene, ketone, fatty acid, alcohol, aldehyde, epoxy, benzene, styrene, diacid, dione, 2-decanone, 2-dodecanone, 2-tetradecanone, 2-hexadecanone, 2-heptadecanone, 2-dotriacontanone, 2.9-decanedione, 2.11-dodecanedione, styrene oxide, α-methyl styrene, toluene, benzoic acid, ethylbenzene, cumyl alcohol, acetophenone, benzaldehyde, benzoic acid, 1-phenylethanol, 2-phenylethanol, 1-phenyl-1,2,3,8a-tetrahydronaphthalene, 1,1-diphenyl ethylene, 2,4-diphenyl-1-butene, trans-1,2-diphenylcyclobutane, 1,3-diphenyl-butene-3, cis-1,2-diphenylcyclobutane, 1-phenyltetralin, 2-phenyltetralin, 1,2-diphenylethane-1,2-dione, (3-phenylbut-3-enyl)benzene, (Z)-1,4-diphenyl-1-butene, 2,4,6-triphenyl-1-hexene, 1,3,5-triphenylcyclohexane, 1,3,5-Triphenylhexene-5, 1-phenyl-4-(1-phenylethyl)tetralin, (1-methyl-2,2-diphenylcyclopropyl) sulfanylbenzene, 2,4-di-tertbutylphenol, 2-ethyl-1-hexanol, tris(2,4-ditert-butylphenyl) phosphite, 1,4,7-trioxacyclotridecane-8,13-dione, 1,6-dioxacyclododecane-7,12-dione, 2,6-di-tert-butyl-pbenzoquinone, and 7,9-Di-tert-butyl-1-oxaspiro[4.5] deca-6,9-diene-2,8-dione are obtained or obtainable by the degradation of the polyolefin and/or polystyrene.

11. (canceled)

12. The isolated enzyme or fragment thereof of claim 2, wherein the enzyme or fragment thereof has an increased ability to degrade the polyolefin and/or polystyrene compared to the corresponding unmodified enzyme.

13. The method of claim 1, wherein the enzyme or fragment thereof, does not comprise a detectable signal sequence, and wherein the enzyme or fragment thereof is secreted out of the cell which produces it.

14. The method of claim 1, wherein the enzyme or fragment thereof is encoded by a heterologous polynucleotide sequence and optionally expressed by a micro-organism or host cell.

15. The micro-organism or host cell of claim 3, wherein the micro-organism or host cell is selected from the group consisting of Escherichia coli, Saccharomyces cerevisiae, Yarrowia lipolytica, Pichia pastoris, Trichoderma reesei, Aspergillus nidulans, Aspergillus niger, Bacillus licheniformis, Bacillus subtilis, and Myceliophthora thermophila.

16. A polynucleotide encoding the enzyme or fragment thereof of claim 2.

17. An expression vector or plasmid comprising the polynucleotide of claim 16.

18. (canceled)

19. A method of producing the enzyme or fragment thereof of claim 2, wherein a recombinant micro-organism or host cell comprising a polynucleotide encoding the enzyme or fragment thereof is allowed to express said enzyme or fragment thereof.

20. The micro-organism or host cell of claim 3, wherein the enzyme or fragment thereof has at least 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 49, and wherein the enzyme originates from or is an enzyme of a bacterium or fungus selected from the group comprising or consisting of Bacillus, Paenibacillus, Achromobacter, Acinetobacter, Alcanivorax, Aneurinibacillus, Arthrobacter, Aspergillus, Brevibacillus, Brucella, Chitinophaga, Citrobacter, Comamonas, Cordyceps, Cupriavidus, Delftia, Enterobacter, Escherichia, Exiguobacterium, Flavobacterium, Fusarium, Halomonas, Hyphomicrobium, Klebsiella, Kocuria, Leucobacter, Lysinibacillus, Macrococcus, Methylobacterium, Methylocella, Microbacterium, Micrococcus, Moraxella, Mucor, Nesiotobacter, Nocardia, Ochrobactrum, Pantoea, Paracoccus, Penicillium, Pleurotus, Pseudomonas, Rahnella, Ralstonia, Rhizobium, Rhodococcus, Serratia, Sphingobacterium, Staphylococcus, Stenotrophomonas, Streptococcus, Streptomyces, Trichoderma, Vibrio, Virgibacillus and Xanthobacter; or the enzyme is an enzyme of a bacterium or fungus selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp, Aspergillus awamori, Aspergillus flavus, Aspergillus fumigatus, Aspergillus glaucus, Aspergillus niger, Aspergillus oryzae, Aspergillus sp. Aspergillus sydowii, Aspergillus terreus, Bacillus amyloliquefaciens, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Brevibacillus sp., Brucella anthropi, Chitinophaga sp., Citrobacter amalonaticus, Comamonas sp., Cordyceps confragosa, Cupriavidus necator, Delftia sp., Delftia tsuruhatensis, Enterobacter asburiae, Enterobacter sp., Escherichia coli, Exiguobacterium sp. Flavobacterium sp., Flavobacterium petrolei, Flavobacterium pectinovorum, Flavobacterium aquicola, Fusarium solani, Fusarium sp., Halomonas venusta, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus luteus, Micrococcus lylae, Moraxella sp., Mucor circinelloides, Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Penicillium chrysogenum, Penicillium oxalicum, Penicillium ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Sphingobacterium multivorum, Staphylococcus epidermidis, Staphylococcus cohnii, Staphylococcus sp., Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Vibrio alginolyticus, Vibrio parahaemolyticus, Virgibacillus halodenitrificans, Xanthobacter autotrophicus, and Xanthobacter tagetidis.

21. The micro-organism or host of claim 3, wherein the micro-organism or host is capable of degrading the polyolefin and/or polystyrene at a temperature of at least 20° C., at least 25° C., at least 30° C., at least 37° C. or at a temperature of 30-95° C.

22. The method of claim 1, wherein the enzyme or fragment thereof is capable of degrading the polyolefin and/or polystyrene at a temperature of at least 20° C., at least 25° C., at least 30° C., at least 37° C. or at a temperature of 30-95° C.

23. The isolated enzyme or fragment thereof of claim 2, wherein at least one or more degradation products selected from the group consisting of an alkane, alkene, alkyne, cycloalkane, alkadiene, ketone, fatty acid, alcohol, aldehyde, epoxy, benzene, styrene, diacid, dione, 2-decanone, 2-dodecanone, 2-tetradecanone, 2-hexadecanone, 2-heptadecanone, 2-dotriacontanone, 2.9-decanedione, 2.11-dodecanedione, styrene oxide, α-methyl styrene, toluene, benzoic acid, ethylbenzene, cumyl alcohol, acetophenone, benzaldehyde, benzoic acid, 1-phenylethanol, 2-phenylethanol, 1-phenyl-1,2,3,8a-tetrahydronaphthalene, 1,1-diphenyl ethylene, 2,4-diphenyl-1-butene, trans-1,2-diphenylcyclobutane, 1,3-diphenyl-butene-3, cis-1,2-diphenylcyclobutane, 1-phenyltetralin, 2-phenyltetralin, 1,2-diphenylethane-1,2-dione, (3-phenylbut-3-enyl)benzene, (Z)-1,4-diphenyl-1-butene, 2,4,6-triphenyl-1-hexene, 1,3,5-triphenylcyclohexane, 1,3,5-Triphenylhexene-5, 1-phenyl-4-(1-phenylethyl)tetralin, (1-methyl-2,2-diphenylcyclopropyl) sulfanylbenzene, 2,4-di-tert-butylphenol, 2-ethyl-1-hexanol, tris(2,4-ditert-butylphenyl) phosphite, 1,4,7-trioxacyclotridecane-8,13-dione, 1,6-dioxacyclododecane-7,12-dione, 2,6-di-tert-butyl-p-benzoquinone, and 7,9-Di-tert-butyl-1-oxaspiro[4.5] deca-6,9-diene-2,8-dione are obtained or obtainable by the degradation of the polyolefin and/or polystyrene.

24. The micro-organism or host cell of claim 3, wherein the micro-organism or host cell has an increased ability to degrade the polyolefin and/or polystyrene compared to the corresponding unmodified micro-organism or host cell.


SEQUENCE LISTING (57 sequences)

SEQ ID NO: 1 - DNA, 1398 bp, Bacillus flexus
  source 1..1398: mol_type = genomic DNA
atgaaaagaacaaaaaagacaattgtatccgttgctctagcaacaagcttagccctttcaatatctagtacatatactgcagctgctcctgctactcaggcttcagcgagtcaagcttacaatgcggtagataaaaaggttctcaacaaaattaatgtagataacatctacaatgatattgaatatttagcaaaaacgcctcgcgtagctggaactgcggctgaacataaagcagcttggtatattcaaaagaaattaaaatcatttggttataaagcaaacgtcgagccatttacattcgttggctatacagaagcatctaaagttgaactttcagctgatgggacaagctattcacctagcgctttcacatatagcataaacggtaatgtaagcgctgaagttgttgatgctggccttggaacaaaagaagagttaacgaatcaagacttgacaggcaaaattgcactgattcaacgtggctctattagctttggtgaaaaagtattaaatgcagctgaaaaaggagccaaagccgttattatttataataatgctgacggtgagttaaacgggacgcttggtggagctaatgatggttatgtaccagctgtagctttaccaaaagcggagggtgaacagcttcttgaaaaaataaaagcaggcaatctaacagctactgtaaatattgaaggtgctgaagttagtgagcgtatttcatataacgtaaccgcaacaaaatcaccaactgaaaaaaaacgcaacacggatgatatcatcgtaattggggctcatcatgactcagttgagggtgcgcctggtgcaaacgacgacgcatctggaacagcaatggtcattgaattagctcgcgtattaaagtctctaccaacagatacggagctacgctttgtaacgtttggagctgaagaacttggcttaattggttctcaagagtacgtggatggtttatctcacgatgagaaagatcgtatcgttggtatgtttaacttagatatggttgggagccgcgatgctggtgatttagtattaaaaacagcagatggaaaagctaactttgtaacaaaattagctcaggcgtcaagcttgcgcctaaatggtgcggctactccatattcagagggtgggcgcagtgatcatgttccgtttgcagaagcaggcattcctgctgcactatttatccatgatccagctgaaccatggtaccatacaccagaagatagcwytgacaaaattagtaaagaaaaacttcaagatgtcgctgaaattgtgggaacygccgtgtatgatcgcgctcgaccagatcataaaggaccaaaaacacaaaaaggaaaaccatttaaagcacctgagctttattatcagcaagatcttaaataa

SEQ ID NO: 2 - protein, 465 aa, Bacillus flexus
MKRTKKTIVSVALATSLALSISSTYTAAAPATQASASQAYNAVDKKVLNKINVDNIYNDIEYLAKTPRVAGTAAEHKAAWYIQKKLKSFGYKANVEPFTFVGYTEASKVELSADGTSYSPSAFTYSINGNVSAEVVDAGLGTKEELTNQDLTGKIALIQRGSISFGEKVLNAAEKGAKAVIIYNNADGELNGTLGGANDGYVPAVALPKAEGEQLLEKIKAGNLTATVNIEGAEVSERISYNVTATKSPTEKKRNTDDIIVIGAHHDSVEGAPGANDDASGTAMVIELARVLKSLPTDTELRFVTFGAEELGLIGSQEYVDGLSHDEKDRIVGMFNLDMVGSRDAGDLVLKTADGKANFVTKLAQASSLRLNGAATPYSEGGRSDHVPFAEAGIPAALFIHDPAEPWYHTPEDSXDKISKEKLQDVAEIVGTAVYDRARPDHKGPKTQKGKPFKAPELYYQQDLK

SEQ ID NO: 3 - DNA, 1350 bp, Bacillus licheniformis
  source 1..1350: mol_type = genomic DNA
atgaagagaaaaatgatgatgttcggattggcgctatcgatcattgcaggcggcgtggtcgccgatggaacggggaatgcagctcaagcggcccctcaggaaacagccatcgcaaaagatatcgaaaaattcagcaaaaaattcaacgagaaccgcgcctatcaaacgatttaccatttaagcgaaacgatcgggccgcgtgtgacaggcacggcagaagaaaaaaagagcgccgcttttatcgcctcacagatgaaaaagtcaaatctgaaagtaagcacacaaaagttcagcatacctgaccggctggaaggaacgcttaccgttcagggaaataacgtgcctgcgcggcctgccgccggttccgccccgactgcagcagaaggcttggccgctcctctctatgatgccggcctcggcttgcccggcgacttcacagaagaagcgaaaggaaaaatcgccgtcattttgagaggagagctgacattctatgaaaaggccaaaaacgctgctgacgcaggcgcaagcggagttatcatttataataacgtcgacagcctcgtccctctgactccgaatctaagcggtaataaagtcgatattccagttgtcggcgtcaaaaaggaagacggggaaaagctgctttctgaacaagaagcgatcctgaagctgaaagctcataaaaatcaaacctcgcaaaacgtaatcggcgtccgcaaagcaaaaggtgtcaaaaatcctgacatcgtttatgtgacttcgcattatgacagcgttccatatgcaccgggagccaatgacaatgcctccggcacttcagtcgtccttgaactggcccgaattttgaagacagtgccagccgacaaagaaattcgctttattacattcggagccgaagaaatcggactcctcggttcgcgccattatgtcagcacattgtcaaatcaggaagtcaaacggagcgttgccaactttaacttagatatggtggcgacaagctgggagaatgcttcacagctgtacatcaatacgcccgacggttcggcaaacctcgtctggcagctaagtaaagccgcttctttaagccttggaaaagacgtattatttttacatcaaggcggatcatccgaccatgtcccattccatgaagccggcatcgactcggccaactttatttggagagagccgggaacaggcgctttggagccttggtaccacaccccttacgacacgattgaacatatcagcaaagacaggytgaaaacagccggtcaaatcgcgggaacagctgtgtataacttcaccaagaaggaaaacagaaaaccgtcttacagctcagttgcccagtaa

SEQ ID NO: 4 - protein, 449 aa, Bacillus licheniformis
MKRKMMMFGLALSIIAGGVVADGTGNAAQAAPQETAIAKDIEKFSKKFNENRAYQTIYHLSETIGPRVTGTAEEKKSAAFIASQMKKSNLKVSTQKFSIPDRLEGTLTVQGNNVPARPAAGSAPTAAEGLAAPLYDAGLGLPGDFTEEAKGKIAVILRGELTFYEKAKNAADAGASGVIIYNNVDSLVPLTPNLSGNKVDIPVVGVKKEDGEKLLSEQEAILKLKAHKNQTSQNVIGVRKAKGVKNPDIVYVTSHYDSVPYAPGANDNASGTSVVLELARILKTVPADKEIRFITFGAEEIGLLGSRHYVSTLSNQEVKRSVANFNLDMVATSWENASQLYINTPDGSANLVWQLSKAASLSLGKDVLFLHQGGSSDHVPFHEAGIDSANFIWREPGTGALEPWYHTPYDTIEHISKDRLKTAGQIAGTAVYNFTKKENRKPSYSSVAQ

SEQ ID NO: 5 - DNA, 1368 bp, Bacillus subtilis
  source 1..1368: mol_type = genomic DNA
atgaaaaagctgctgaccgttatgaccatggcggttctgaccgcgggtaccgtgctgctgccggcgcagagcgtgaccccggcggcgcgtgcggtgcagattagcgacagcgaacgtgagctgccgttcaaaggtaaacgcgcgtacagcaccattagccaactgagcgaagcgatcggtccgcgcatcgcgggtaccgcggcggaaaagaaaagcgcgctgctgatcgcgagcaccctgcgtaaactgaagctggatgttaaagtgcagcgtttcagcattccggaccgtctggaaggtaccctgagcagcgaaggtcgtgatatcctgctgcgtgcggcgagcggtagcgcgccgaccgaggaaaagggtctgaccgcgccgctgtacaacgcgggtctgggttatccgaaagatttcaccgcggacgcgaagggtaaaatcgcgctgattgcgcgtggtgatctgaccttctacgagaaagcgaagaacgcggaagacgcgggcgcgaaggcggtgatcatctacaataacaaggagagcctggtgccggtgaccccgaacctgagcggcaataaagcgggtattccggttgttggcatcaagaaagaggacggtgaagcgctggcgcagcaaaaagaggcgaccctgaaactgaacgcgttcgcgaatcagaccagccaaaacattatcggcattaagaaaccgaagaacatcaaacacccggatatcgtttacgtgaccgcgcattatgatagcgtgccgttcagcccgggtgcgaatgacaacggtagcggcaccagcgtgatgctggaaatggcgcgtgtgctgaaaagcgttccgagcgacaaggagatccgtttcatcgcgtttggtgcggaggaactgggtctgctgggcagcagccactacgttgaccacctgagcgagaaagaactgaagcgtagcgaagtgaacttcaacctggacatggttggtaccagctgggaaaacgcgagcgagctgtacgtgaacaccctggatggtcagagcaacgaagtgtgggagagcagccacaccgcggcggagaagatcggtttcgatagcctgagcctgacccaaggcggtagcagcgatcatgtgccgtttcacgaggcgggtatcgacagcgcgaacttcatctggggcgatccggaaaccgaggaagttgagccgtggtaccacaccccggaggacagcatcgagcacattagcaaggaacgtctgcaacaggcgggtgatctggttaccgcggcggtttaccaagcggtgaaaaaggagaagaaaccgaagaccaataaaaagcaaatgaaggcgaaagcgagcgacatctacgaagacatcaagtaa

SEQ ID NO: 6 - protein, 455 aa, Bacillus subtilis
MKKLLTVMTMAVLTAGTVLLPAQSVTPAARAVQISDSERELPFKGKRAYSTISQLSEAIGPRIAGTAAEKKSALLIASTLRKLKLDVKVQRFSIPDRLEGTLSSEGRDILLRAASGSAPTEEKGLTAPLYNAGLGYPKDFTADAKGKIALIARGDLTFYEKAKNAEDAGAKAVIIYNNKESLVPVTPNLSGNKAGIPVVGIKKEDGEALAQQKEATLKLNAFANQTSQNIIGIKKPKNIKHPDIVYVTAHYDSVPFSPGANDNGSGTSVMLEMARVLKSVPSDKEIRFIAFGAEELGLLGSSHYVDHLSEKELKRSEVNFNLDMVGTSWENASELYVNTLDGQSNEVWESSHTAAEKIGFDSLSLTQGGSSDHVPFHEAGIDSANFIWGDPETEEVEPWYHTPEDSIEHISKERLQQAGDLVTAAVYQAVKKEKKPKTNKKQMKAKASDIYEDIK

SEQ ID NO: 7 - DNA, 1368 bp, Bacillus cereus
  source 1..1368: mol_type = genomic DNA
atgaagaaactgctgaccgtgatgaccatggcggtgctgaccgcgggcaccgttctgctgccggcgcaaagcgttaccccggcggcgcacgcgattcagatcagcgatagcgagcgtgagctgccgtttaaagcgaagcgtgcgtatagcaccattagccagctgagcgaagcgattggtccgcgtattgcgggtaccgcggcggaaaagaaaagcgcgctgctgatcgcgagcagcctgcgtaaactgaagctggatgtgaaagttcaacgtttcggtattccggaccgtctggaaggtaccctggcgagcgagggccgtgacattctgctgcgcgcggcgagcggcagcgcgccgaccgatgaacaaggtctgaccgcgccgctgtataacgcgggtctgggctacccgaaagatttcaccgcggatgcgaagggtaaaatcgcgctgattagccgcggtgacctgacctattacgagaaggcgaaaaacgcggaggacgcgggcgcgaaggcggtgattatctacaacaataaggagagcctggtgccggttaccccgaatctgagcggcaacaaagcgggtatcccggttgtgggtatcaagaaagaggacggcgaagcgctggcgcaacagaaggaagcgaccctgaagctgaaagcgttcagcaaccaaaccagccagaacattatcggtatcaagaagccgaagaacatcaaacatccggacatcgtgtatgttaccgcgcactacgatagcgttccgtttagcccgggtgcgaacgacaacggtagcggcaccagcgttatgctggaaatggcgcgtgttctgaagagcgtgccgagcgataaggagattcgtttcatcgcgtttggtgcggaagagctgggtctgctgggtagcagccactatgtggaccacctgagcgaaaaagagctgaagcgtagcgaggtgaactttaacctggatatggtgggtaccagctgggaaaacgcgagcgagctgtacgttaacaccctggacggtcagagcaacgatgtgtgggaaagcagccacaccgcggcggagaagatcggtttcgatagcctgagcctgacccaaggtggcagcagcgatcatgttccgtttcacgaagcgggtatcgatagcgcgaacttcatctggggtgatccggaaaccgaggaagttgaaccgtggtaccacaccccggaagacagcatcgaacacatcagcaaggagcgtctgcagcaagcgggtgacctggttaccgcggcggtgtaccaggcggttaagaaagagaagaaaccgaagaccatcaaaaagcagatgaaagcgaaggcgagcgacatctacgaggatatcaaataa

SEQ ID NO: 8 - protein, 455 aa, Bacillus cereus
MKKLLTVMTMAVLTAGTVLLPAQSVTPAAHAIQISDSERELPFKAKRAYSTISQLSEAIGPRIAGTAAEKKSALLIASSLRKLKLDVKVQRFGIPDRLEGTLASEGRDILLRAASGSAPTDEQGLTAPLYNAGLGYPKDFTADAKGKIALISRGDLTYYEKAKNAEDAGAKAVIIYNNKESLVPVTPNLSGNKAGIPVVGIKKEDGEALAQQKEATLKLKAFSNQTSQNIIGIKKPKNIKHPDIVYVTAHYDSVPFSPGANDNGSGTSVMLEMARVLKSVPSDKEIRFIAFGAEELGLLGSSHYVDHLSEKELKRSEVNFNLDMVGTSWENASELYVNTLDGQSNDVWESSHTAAEKIGFDSLSLTQGGSSDHVPFHEAGIDSANFIWGDPETEEVEPWYHTPEDSIEHISKERLQQAGDLVTAAVYQAVKKEKKPKTIKKQMKAKASDIYEDIK

SEQ ID NO: 9 - DNA, 86 bp, synthetic construct
  source 1..86: mol_type = other DNA
  misc_feature 1..86: note = oPlastBug-134 oligonucleotide
acaattcctctagaaataattttgtttaactttaagaaggagatatatccatgaaaagaacaaaaaagacaattgtatccgttgct

SEQ ID NO: 10 - DNA, 86 bp, synthetic construct
  source 1..86: mol_type = other DNA
  misc_feature 1..86: note = oPlastBug-135 oligonucleotide
ttgttagcagccggatcaagctgggatttaggtgacactatagaatactcttatttaagatcttgctgataataaagctcaggtgc

SEQ ID NO: 11 - DNA, 82 bp, synthetic construct
  source 1..82: mol_type = other DNA
  misc_feature 1..82: note = oPlastBug-124 oligonucleotide
acaattcctctagaaataattttgtttaactttaagaaggagatatatccatgaagagaaaaatgatgatgttcggattggc

SEQ ID NO: 12 - DNA, 76 bp, synthetic construct
  source 1..76: mol_type = other DNA
  misc_feature 1..76: note = oPlastBug-125 oligonucleotide
ttgttagcagccggatcaagctgggatttaggtgacactatagaatactcttactgggcaactgagctgtaagacg

SEQ ID NO: 13 - protein, 434 aa, Bacillus amyloliquefaciens
MLIRGGIMMKFSFMSAAVAASVIWGASAGPLSETHAYSAVTAPVFDADEALDTMHDLSETIGPRETGTAKEKEAAEYISNRLRAQGASVKVQSFPLKKYFSGSVTAGDHEFPLRSADQSAVEKELRIEGGIFDGGCGSRDDVTGAKGKIAVIREGKIPVSQKIKHAEQAGAKAVIIYNQTTKPLAVRPHTDKGITIPAAGMKKEDGEALLQEKQAVIQFKNYKNRTSQNIIGTRKAPQANSSDADIIYVTAHYDSVKGAPGADDNASGTSAVLEISKQLTHIPKDKEIRFILFGAEELWSAGSSYYVSTLTNNERKHSQANFNADMIATDWEEASQMCVTTPDGKENPSVRYAEKAAEKSRLVKPNLIQLGYSDHVSFYEAGIPSANWIWLSPDTQEPGPWYHTENDNMKHISKERLEKAGSLVSAAVTEAVKE

SEQ ID NO: 14 - protein, 465 aa, Bacillus cereus
MRKSLKQKIVSSLLTVSLAFSLAPIVQAKADSTSEITQAPSITKQVDASRAIEHIRFLSETIGPRPGGTKSEEWASRYVGMQLKSMGYEVEYQPFQVPDQYVGFIESPLSTKRNWQAGAAPNALISTAAITAPLIFVKNGTKLDEIPNEVSGKIVLFERGATVADYNKQVENAVSKGAKGVLLYSLIGGRGNYGQTFNPRLTKKQSVPVFGLAYAQGNAFKEEIAKKGTTILSLKARHESNLTSLNVIAKKKPKNSTGNEKAVVVSSHYDSVGGAPGANDNASGTGLVLELARAFQNVETDKEIRFIAFGSEEMGLLGSGYYVNSLSQKERDRILGVFNADMVATNYDKAKNLYAMTPDGSTNLVTDAALQAGKQLNNDLVLQGKFGSSDHVPFAEVGIPSALFIWMGVDSWNPLIYHIEKVYHTPQDNVFENISPERMKMALEVIGTGVYDILQSNVKAGQKAA

SEQ ID NO: 15 - protein, 452 aa, Bacillus sp.
MKVGRKASSIMLAAVLTASSLYAVPVQFVQSAEAAQSKALDPGSKAFDQKVIARVDASRMMEDVSYLSETIGPRVAGTEAEKQAANFIRKRLESYGYTVETQEFSIPDKMAGDLHTSDLKEVLITIPSGSGSTPEEGITSELYDAGLGRAADFTEEAKEKIALISRGEISFAEKVTNAVNAGAAGVLIYNNVDQPAPMNPSIGGPYDIPVGSITKASGEALLQDVAAHNKTVTLTVKSFKNIRSQNIIATKHPKPNKGGETDIVHISAHFDSVPFAPGASDNASGTAVALELARVLKSYPADKELRFAFVGAEEIGLLGSQYYVSQLSGNEIERSIANFNMDMVGTAWENATAIYMNTLDGQANIATEAAIATAGRIGTPSELVLYQRGASDHVSFHDAGIPAVNFIRREPATANLEPYYHTPLDSIEHISAERMKEAGDLIGASVYSLIRK

SEQ ID NO: 16 - protein, 427 aa, Bacillus sp.
MMKFSFMSAAVAASVIWGASAGPLSETHAYSAVTAPVFDADEALDTMHDLSETIGPRETGTAKEKEAAEYISNRLRAQGASVKVQTFPLKKYFSGSVTAGDHEFPLRSADQSAVEKELRIEGGIFDGGCGSRNDVTGAKGKIAVIREGKIPVSQKIKHAEQAGAKAVIIYNQTTKPLAVRPHTDKGITIPAAGMKKEDGEALLQEKQAVIQFKNYKNRTSQNIIGTRKAAQANSSDADIIYVTAHYDSVKGAPGADDNASGTSAVLEISKQLKHIPKDKEIRFILFGAEELWSAGSSYYVSTLTNNERKHSQANFNADMIATDWEEASQMCVTTPDGRENPSVRYAEKAAEKSRLVKPKLIQLGYSDHVSFYEAGIPSANWIWLSPDTQEPGPWYHTENDNMKHISKERLEKAGALVSAAVTEAVKE

SEQ ID NO: 17 - protein, 426 aa, Bacillus sp.
MKFSFMSAAVAASVIWGASAGPLSETHAYSAVAAPVFDADEALDTMHDLSETIGPRETGTAKEKEAAEYISNRLRAQGASVKVQSFPLKKYFSGSVTAGDHEFPLRSADQSAVEKELRIEGGIFDGGCGSRDDVTGAKGKIAVIREGKIPVSQKIKHAEQAGAKAVIIYNQTTKPLAVRPHTDKGITIPAAGMKKEDGEALLQEKQAVIQFKNYKNRTSQNIIGTRKAAQANSSDADIIYVTAHYDSVKGAPGADDNASGTSAVLEISKQLKHIPKDKEIRFILFGAEELWSAGSSYYVSTLTNNERKHSQANFNADMIATDWEEASQMCVTTPDGKENPSVRYAEKAAEKSRLVKPNLIQLGYSDHVSFYEAGIPSANWIWLSPDTQEPGPWYHTENDNMKHISKERLEKAGALVSAAVTEAVKE

SEQ ID NO: 18 - protein, 455 aa, Bacillus subtilis
MKKLLTVMTMAVLTAGTLLLPAQSVTPAAHAVQISNSERELPFKAKHAYSTISQLSEAIGPRIAGTAAEKKSALLIASSMRKLKLDVKVQRFNIPDRLEGTLSSAGRDILLQAASGSAPTEEQGLTAPLYNAGLGYQKDFTADAKGKIALISRGDLTYYEKAKNAEAAGAKAVIIYNNKESLVPMTPNLSGNKVGIPVVGIKKEDGEALTQQKEATLKLKAFTNQTSQNIIGIKKPKNIKHPDIVYVTAHYDSVPFSPGANDNGSGTSVMLEMARVLKSVPSDKEIRFIAFGAEELGLLGSSHYVDHLSEKELKRSEVNFNLDMVGTSWEKASELYVNTLDGQSNYVWESSRTAAEKIGFDSLSLTQGGSSDHVPFHEAGIDSANFIWGDPETEEVEPWYHTPEDSIEHISKERLQQAGDLVTAAVYEAVKKEKKPKTIKKQMKAKASDIFEDIK

SEQ ID NO: 19 - protein, 466 aa, Bacillus sp.
MRKPILKVALASTLIFGAIGTQAVFAHPSDVKQTQPVNVFDKKVVNQINAENIYNNIDYLSQTPRVAGTESEYNAVQYIKSQFESYGYETEIQPFTFYGYTPPSSMEVTIDGYGGELQPRSFTYSVNGDVSGELAFAGLGTQEELEGVDLTGKIALIQRGSISFAEKVLNAAEKGAAGVVIFNNAEGPLSGTLGAHNEEYVPALALSKAEGEAILALLESGETLSASLSIEGADAGEKVSHNVIATKKPTNNKKANDDIIVVGSHHDSVAGAPGANDDASGTAMTLELARVFKDIPTDTEIRFATFGAEELGLIGSYHYVDSLSDDEISRTIANFNLDMVGSKDAGDLVMMTLDGEENLVTELSQASSERLNGEPTPVYQGGRSDHVPFAEAGIAAALFIHSPTEPWYHSPEDTIDKISKEKLQDVAEVVGAAIYDKARFDNMGPKPKKQHKMKAAPEMVHELDVK

SEQ ID NO: 20 - protein, 464 aa, Bacillus sp.
MKKTFVSMSLAGVLAFGAVHAPALAQPKEAPKLEAIHGLENSLIKRISAEKIYQSITDLSQSPRVAGTASEDQAVQYIKQKFESYGYKTEIQPFTFYGYTPPEKIELTVQGAEEPLSPAPFTYTVNGNVTAELAEAGLGTKEDAAAADLEGKIALIQRGEISFAEKVLNAAEQGAAGVLIYNNADGPLSGTLGEANEEYIPAAALSKAEGESLSARLAEGETLTANLVIEGADAGEKTSHNVIAVKKPTNKKKDTGEVIVLGAHHDSVAGAPGANDDASGTAMTLELARVFKNIPTDSEIRFVTFGAEELGLLGSRHYVENLSDKEQDSIIANFNLDMVGSRDAGDLVMLTADGEPNLVTELAQKSSLKLNGTATPYGQGGRSDHVPFAEAGIPAALFIHSPSEPWYHTPEDTIDKISKDKLQDVAEIVGSAIYDQAKFDRKLPKPKKGKKHKTPHLYQEQEVK

SEQ ID NO: 21 - protein, 471 aa, Bacillus cereus
MGEEYMKKTWKQKIVSSLLAATLAVSLAPIGQVKADSPQTTIETPPITKQVDTNRAIEHVRFLSETIGPRPGGTQSEKWAARYVGMKLQSMGYEVEYQPFAVPDQYVGFIESPLSKSRNWQTGAAPNSLISTEAVTAPLIFVPNGTNLDEVPNEVNGKIVLFERGATVADYNKQVENAVAKGAKGVLLYSLIGGRGNYGQTFNPRLTKKQSIPVFGLAYAQGNAFKEELAKKGDTILSLKARHESNLQSLNVIAKKKPKNSTGNEKAVIVSSHYDSVAGAPGANDNASGTGLVLELARAFQNVETDKEIRFIAFGSEEMGLIGSEHYVDNLSQKERDRILGVFNADMVATNYDKAKNLYAMTPDGSTNLVTDATLHAAKQLNNDLVLQGKFGSSDHVPFAYAGIPAALFIWMGVDSWNPLIYHIEKVYHTPQDNVLENISPERMKMALDVIGTGVYDVLQKPVVQSGQKAA

SEQ ID NO: 22 - protein, 466 aa, Bacillus mycoides
MKRTLKQKIVSSLLAATFAVSLAPIGQAKADSPSETIKTPPITGQVNANRAIEHVRFLSETIGPRPGGTQSEKWASRYVGMQLQSMGYEVEYQPFAVPDQYVGFIDSPLSRSKNWQAGAAPNALISTEAVTAPLIFVPNGTKIEEIPNEVNGKIVLFERGATVADYNKQVENAVAKGAKGVLLYSLIGGRGNYGQTFNPRLTKKQSIPVFGLAYAQGNAFKEELAKKGSTILSLKARHESNLQSLNVIAKKKPKKSTGNEKAVIVSSHYDSVAGAPGANDNASGTGLVLELARAFQNVETDKEIRFIAFGSEEMGLLGSEHYLDNLSQKERDRILGVFNADMVATNYDKAKNLYAMTSDGSTNLVTDAALNASKQLNNDLVLQGKFGSSDHVPFAYAGIPAALFIWMGVDSWDPLIYHIEKVYHTPQDNVLENISPERMKMALDVIGTGVYDVLQKPAVQTGQKAA

SEQ ID NO: 23 - protein, 458 aa, Bacillus sp.
MSRNSRKSPKILAAALAASLAFGTIGYAAPLSNENGNSSHSQDQKIIARVNAERAIEHVRHLSEEIGTRPGGLENEKKSAEYIANTLKSYGYDVEFQYFPVADQFIGSAAFEDGTVWEMGAAPNGAISDTPVHAEVIFVENENFQGAEGKIVLLARADTTAGYREQVAKAVEAGAAGVILQSLVGSRGNYGQTFNPNLTSEYDIPVFGASYIHGEWLKEQMEQGAVELSLSAKHYKDLKSVNVIATKEAKSKDEDTKEVILGAHHDSVVGAPGANDNASGVGLMLELARVYKGYNTDKTLKFIAFGSEERGLLGARYYVDQLTETQKDQIEAVFVPDMVATNYEKATNLYAMTPDGSQNIVTSSTGQAGARLGNSDILPGKFGSSDHVPFHNAGIPAALFIWMGIDSWDPLVYHIEKVYHTPQDTIEDNISAERMQSALDIIGSGLFDVVRKKTQGNN

SEQ ID NO: 24 - protein, 466 aa, Bacillus sp.
MRKSLKQKIVSSLLAVSLAVSLAPIGQAKADSTSEVTQTPSITKQIDPNRAIEHVRFLSETIGPRPGGTKSEEWASRYIGMQLKSMGYEVEYQPFQVPDQYVGFIESPLSTKRNWQAGAAPNALISTEAVTAPLIFVQGGTKLEDIPNEVNGKIVLFERGTTVTDYNKQVENAVSKGAKGVLLYSLIGGRGNYGQTFNPRLMKKQSIPVFGLAYAQGNAFKEEIAKKGTTILSLKARHESKLTSLNVIAKKKPKKSTGNEKAVVVSSHYDSVVGAPGANDNASGTGLVLELARTFQNVETDKEIRFITFGSEETGLLGSDYYVNSLSQKERDRILGVFNADMVATNYDKAKNLYAMTHDGSTNFVTDAALQAGKQLNNNLILQGKFGSSDHVPFAEAGIPAALFIWMGVDSWNPLIYHIEKVYHTPQDNVLENISPERMRMALDVIGTGVYNSLQKPVTQTEQKAA

SEQ ID NO: 25 - protein, 455 aa, Bacillus sp.
MKKLLTVMTMAVLTAGTLLMPAQSVTPAAHAVQLSDSERELPFKAKHAYSTISQLSEAIGPRIAGTAAEKKSALLIASSMRKLKLDVKVQRFDIPDRLEGTLSSEGRDILLQAASGSAPTEEQGLTAPLYNAGLGYQKDFTADAKGKIALISRGDLTYYEKAKNAQAAGAKAVIIYNNKESLVPMTPNLSGNKAGIPVVGIKKEDGEALTQQKEATLKLKAFTNQTSQNIIGIKKPKNIKHPDIVYVTAHYDSVPFSPGANDNGSGTSVMLEMARVLKSVPSDKEIRFIACGAEELGLLGSSHYVDHLSEKELKRSEVNFNLDMVGTSWEKASELYVNTLDGQSNAVWESSHTAAEKIGFDSLSLTQGGSSDHVPFHEAGIDSANFIWGDPETEEVEPWYHTPEDSIEHISKDRLQQAGDLVTAAVYEAVKKEKKPKTIKKQMKAKASDIYEDIK

SEQ ID NO: 26 - protein, 466 aa, Bacillus subtilis
MKKSLKQKIVSSLLAVSLAVSLAPIGQAKADSTSEIKQTSPITKQVDASRAIEHIRFLSETIGPRPGGTKSEEWASRYVGMQLKSMGYEVEYQPFQVPDQYVGFIESPLSTKRNWQAGAAPNALISTESVTAPLIFVQGGTKLEDIPNEVNGKIVLFERGTTVADYNKQVENAVSKGAKGVLLYSLIGGRGNYGQTFNPRLTKKQSIPVFGLAYAQGNAFKEEIAKKGTTILSLKARHESNLTSLNVIAKKKPKNSTGNEKAVVVSSHYDSVVGAPGANDNASGTGLVLELAHAFQNVETDKEIRFIAFGSEETGLLGSDYYVNSLPQKERDRILGVFNADMVATNYDKAKNLYAMTPNGSPNLVTDAALQAGKQLNNDLVLQGKFGSSDHVPFAEVGIPAALFIWMGVDSWNPLIYHIEKVYHTPQDNVFENISPERMKMALEVIGTGVYNTLQQPATQTEQKAA

SEQ ID NO: 27 - protein, 465 aa, Bacillus cereus
MKKSLKQKIVSSLLAVSLAVSIAPVGQAKADSTSEITQTSPITKQVDASRAIEHIRFLSETIGPRPGGTKSEEWASRYVGMQLKSMGYEVEYQPFQVPDQYVGFIESPLSTKRNWQAGAAPNALISTEAVTAPLIFVQDGTKLEDIPNEVNGKIVLFERGTTVADYNKQVENAVSKGAKGVLLYSLIGGRGNYGQTFNPRLTKKQSIPVFGLAYAQGNAFKEEIAKKGTTILSLKARHESNLTSLNVIAKKKPKNSTGNEKAVVVSSHYDSVVGAPGANDNASGTGLVLELARAFQNVETDKEIRFIAFGSEETGLLGSDYYVNSLPQKERNRILGVFNADMVATNYDKAKNLYAMTPNGSPNLVTDAALQAGKQLNNELVLQGKFGSSDHVPFAEVGIPAALFIWMGVDSWNPLIYHIEKVYHTPQDNVLENISLERMKMALEVIGTGVYNSLHSTVKTGQKAA

SEQ ID NO: 28 - protein, 457 aa, Bacillus cereus
MKKLTLIKKLIVTFGILLLFLFSNLSEDVILVHAMTKNDQSSLMSDKVRASRAIEHTRFLSEKIGPRPSGTKEEHKAATYIGDTLKKLGYEVEYQAFPVSDQYIGSISFSVYGEKEWQVGAASNASIPKDPVVGPVVSIGKENDIKNSPEPIHGKIVLLEQADTYEKYNEQVEYAVEKGAKAVLLYSTIGDRGNYGEAFNPVLKKKQSIPVFGLAYNQGIRMKDKIVHNKNIVSLKAKHEMNLSSLNVIAKKKPKYKTDNEDVAIMSAHYDSVIGAPGANDNASGVGLLLELAYNFKDIEINKEIQFIAFGAEENESLGAYHYVNQLSTTDKKHILGVFNADMIATSYQQAKDLYAMTLDGSQNEVTNTAVATSKALGKSVVLTGKFDSSDHVPFYEAGISSALFIWMDVESWNPLIYHVEKVFHTPQDTVLENISPQRMQEALEIIGTSLYYFIKR

SEQ ID NO: 29 - protein, 479 aa, Bacillus cereus
MSVRENRRIGEEKMRKSLKQKIVSSLLAVSLAVSLAPIGQAKADSTSEVTQTPSITKQIDPNRAIEHVRFLSETIGPRPGGTKSEEWASRYIGMQLKSMGYEVEYQPFQVPDQYVGFIESPLSTKRNWQAGAAPNALISTEAVTAPLIFVQGGTKLEDIPNEVNGKIVLFERGTTVTDYNKQVENAVSKGAKGVLLYSLIGGRGNYGQTFNPRLMKKQSIPVFGLAYAQGNAFKEEIAKKGTTILSLKARHESKLTSLNVIAKKKPKKSTGNEKAVVVSSHYDSVVGAPGANDNASGTGLVLELARAFQNVETDKEIRFITFGSEETGLLGSDYYVNSLSQKERDRILGVFNADMVATNYDKAKNLYAMTPDGSTNFVTDAALQAGKQLNNNLILQGKFGSSDHVPFAEAGIPAALFIWMGVDSWNPLIYHIEKVYHTPQDNVLENISPERMRMALDVIGTGVYNSLQKPVPQTEQKAA

SEQ ID NO: 30 - protein, 457 aa, Bacillus cereus
MKKLAITKKIIVSFGILLLFFFSNPYGEPVKVNAMVNNNLYSPVTEQVRASRAIEHIRFLSEKIGPRPSGTMQEHRAAKYIGDTLQKLGYEVEYQALPVPDQYIGSISFSMYGEKDWQVGAASNAFISKNPVVGSVVFIGKETNINSLSESIHGKIVLLEQADTIEKYNEQVEHVAIKGAKGVLLYSTIGDRGNYGEAFNPILKKEQSIPVFGLAYNQGIRMKEKLLHNQNVVSLKAKQEVNLRSLNVIAKKKPKHTTGDEEVAIMSAHYDSVIGAPGANDNASGVGLLLELAYNFKDIETNKEIQFIAFGAEENESLGAYHYVNQLSDKDKTRIRGVFNADMIATSYHQAKDLYAMTLDGSQNEVTNTAVATSKELRKSVVLTGKFDSSDHVPFNQAGIPSALFIWMDVESWDPLIYHVERVFHTPQDTVLENISAQRMQDALEIIGTSLYYFIKR

SEQ ID NO: 31 - protein, 457 aa, Bacillus cereus
MKKLTLIKKLIVTFGILLLFLFSNLSEDVILVHAMTKNDQSSLMSDKVRASRAIEHTRFLSEKIGPRPSGTKEEHKAATYIGDTLKKLGYEVEYQAFPVPDQYIGSISFSVYGEKEWQVGAASNASIPKDPVVGPVVSIGKENDIKNSPEPIHGKIVLLEQADTYEKYNEQVEYAVEKGAKAVLLYSTIGDRGNYGEAFNPVLKKKQSIPVFGLAYNQGIRMKDKIVHNKNIVSLKAKHEMNLSSLNVIAKKKPKYKTDNEDVAIMSAHYDSVIGAPGANDNASGVGLLLELAYNFKDIEINKEIQFIAFGAEENESLGAYHYVNQLSTTDKKHILGVFNADMIATSYQQAKDLYAMTLDGSQNEVTNTAVATSKALGKSVVLTGKFDSSDHVPFYEAGISSALFIWMDVESWNPLIYHVEKVFHTPQDTVLENISPQRMQEALEIIGTSLYYFIKR

SEQ ID NO: 32 - protein, 466 aa, Bacillus cereus
MKRTLKQKIVSSLLAATLAVSLAPIGQAKADSPSETIKTPPITGQVDANRAIEHVRFLSETIGPRPGGTQSEKWASRYVGMKLQSMGYEVEYQPFAVPDQYVGFIDSPLSRSKNWQAGAAPNALISTEAVTAPLIFVPNGTNLDEIPNEVNGKIVLFERGETVTDYNKQVENAVAKGAKGVLLYSLIGGRGNYGQTFNPRLTKKQSIPVFGLAYAQGNAFKEELTKKGNAILSLKARHESNLQSLNVIAKKKPKNSTGNEKAVIVSSHYDSVAGAPGANDNASGTGLVLELARAFQNVETDKEIRFIAFGSEEMGLIGSEHYVDNLSQTERDRILGVFNADMVATSYKKAKNLYAMTSDGSTNLVTDAALNASKQLNNDLVLQGTFGSSDHVPFAYAGIPAALFIWMGVDSWDPLIYHIEKVYHTPQDNVLENISPERMKMALDVIGTGVYDVLQKPAIQTGQKAA

SEQ ID NO: 33 - protein, 466 aa, Bacillus cereus
MRKSLKQKIVSSLLAVSLAVSLAPIGQAKADSTSEVTQTPSITKQIDPNRAIEHVRFLSETIGPRPGGTKSEEWASRYIGMQLKSMGYDVEYQPFQVPDQYVGFIESPLSTKRTWQAGASPNALISTEAVTAPLIFVQGGTKLEDIPNEVNGKIVLFERGTTVTDYNKQVENAISKGAKGVLLYSLIGGRGNYGQTFNPRLTKKQSIPVFGLAYAQGNAFKEEIAKKGTTILSLKARHESKLTSLNVIAKKKPKKSTGNEKAVVVSSHYDSVVGAPGANDNASGTGLVLELARAFQNVETDKEIRFITFGSEETGLLGSDYYVNSLSPKERDRILGVFNADMVATNYDKAKNLYAMTPDGSTNFVTDAALQAGKQLNNELVLQGKFGSSDHVPFAEVGIPAALFIWMGVDSWNPLIYHIEKVYHTPQDNVLENISPERMRMALDVIGTGVYNSLQKPVPQTEQKAA

SEQ ID NO: 34 - protein, 425 aa, Bacillus subtilis
MKMILMSAALAGSVIWGSSAVQVNGIQSHSISEMAPLFDEEHAYETIHELSEVIGPRETGTEKEKEAASYISKRIQDEGLAVRIQPFHVKTYYSGSVMAGDHEYPIRSADKSAVENEERVEGEVLDGGYGLNLDEKEAKGKIAVLREGKISVSQKLKNAQAAGAKTVIIYNSKTKPLAIRPVKPKHITIPAAGMKKEDGEALLKEKQAVIQFKNYRNRTSQNIIGTRKAQHTKHPDILYVTAHYDTVKDAPGADDNASGTSAVLEMIEQSKHLPKNKEIRFIFFGAEELWSAGSSYYVSTLTSDEIKHSRANFNADMIATDWKDASQMYITTPDGKKNASVNYAVNAAQRAQLKAPRLTQLGYSDHVSFYEAGIPSANWIWLSPDTQEPGPWYHTEKDNIAHISKERLNEAGQLLLSAVQEANID

SEQ ID NO: 35 - protein, 466 aa, Bacillus thuringiensis
MSKSLKQKIVSSLLAISLAVSLAPIGQAKADSTSEVPQTPSITKQIDPNRAIEHVRFLSETIGPRPGGTKSEEWASRYIGMQLKSMGYDVEYQPFQVPDQYVGFIESPLSTKRNWQAGAAPNALISTEAITAPLIFVQGGTKLEDIPNEVKGKIVLFERGTTVTDYNKQVENAVSKGAKGVLLYSLIGGRGNYGQTFNPRLTKKQSIPVFGLAYAQGNAFKEEITKKETTILSLKARHESKLTSLNVIAKKKPKKSTGNEKAVVVSSHYDSVVGAPGANDNASGTGLVLELARAFQNVEIDKEIRFITFGSEETGLLGSDYYVNSLSQKERDRILGVFNADMVATNYDKAKNLYAMTPDGSTNFVTDAALQAGKQLNNDLVLQGKFGSSDHVPFAEAGIPAALFIWMGVDSWNPLIYHIEKVYHTPQDNVLENISPERMKMALDVIGTGVYNSLQKPITQTEQKAA

SEQ ID NO: 36 - protein, 479 aa, Bacillus thuringiensis
MSVRENRRIGEEKMKKSLKQKIVSSLLAVSLAVSLAPIGQAKADSTSEIKQTSSITKQVDASRAIEHIRFLSETIGPRPGGTKSEEWASRYVGMQLKSMGYEVEYQPFQVPDQYVGFIESPLSTKRNWQAGAAPNALISTESVTAPLIFVQGGTKLEDIPNEVSGKIVLFERGTTVADYNKQVENAVSKGAKGVLLYSLIGGRGNYGQTFNPRLTKKQSIPVFGLAYAQGNAFKEEIAKKGTTILSLKARHESNLTSLNVIAKKKPKNSTGNEKAVVVSSHYDSVVGAPGANDNASGTGLVLELARAFQNVETDKEIRFIAFGSEETGLLGSDYYVNSLSQKERDRILGVFNADMVATNYDKAKNLYAMTPNGSPNLVTDAALQAGKQLNNDLVLQGKFGSSDHVPFAEVGIPAALFIWMGVDSWNPLIYHIEKVYHTPQDNVFENISPERMKMALEVIGTGVYNTLQQSVTQTEQKAA

SEQ ID NO: 37 - protein, 479 aa, Bacillus thuringiensis
MSVRENRGIGEEKMSKSLKQKIVSSLLAISLAVSLAPIGQTKADSTSEVTQTSSITKQIDPNRAIEHVRFLSETIGPRPGGTKSEEWASRYIGMQLKSMGYEVEYQPFQVPDQYIGFIESPLSTKRNWQAGAAPNALISTEAITAPLIFVQGGTKLEDIPNEVNGKIVLFERGTTVTDYNKQVENAVSKGAKGVLLYSLIGGRGNYGQTFNPRLTKKQSIPVFGLAYAQGNAFKEEIAKKETTILSLKARHESKLTSLNVIAKKKPKKSTGNEKAVVVSSHYDSVVGAPGANDNASGTGLVLELARAFQNVETDKEIRFITFGSEETGLLGSDYYVNSLSQKERDRILGVFNADMVATNYDKAKNLYAMTPDGSTNFVTDAALQAGKQLNNDLVLQGKFGSSDHVPFAEAGIPAALFIWMGVDSWNPLIYHIEKVYHTPQDNVLENISPERMKMALDVIGTGVYNSLQKSITQTEQKAA

SEQ ID NO: 38 - protein, 457 aa, Bacillus thuringiensis; identical to SEQ ID NO: 31

SEQ ID NO: 39 - protein, 307 aa, Bacillus thuringiensis
MREPIAPGGTNDTENELLISGSFHNHSTLVNSIQSLVNQVSENQFQSYLTHLVSYPTRFSTSSFYEEAATWTREILNEMGYTTSSETIRVNNEDSQNVIANKMGTNRDEEKHLIIITAHLDSVNHSGGPSSNAPGADDNGSGSAGVLEIARVFKDIPVQHDLRFILFGGEEQGLLGSKQYVENLPTPDKNRIRAVINMDMIGKLNTSTPAVLIEGSPLSEELINGLKNVASLYTDLDVKVSLNPFDSDHVSFLNKGIPAVLTIEGEDSLNPNDHTANDTLEHIHYPLALDILRMNIAYIASQVTNLE

SEQ ID NO: 40 - protein, 307 aa, Bacillus thuringiensis
MKTSIFPGGTNDSHNELHVSDNFPNHSSPVNSIQSLVNELSENQFRSYLTHLVSYPTRFSTSSFYEEAATWARKILDEMGYKTSSETISVNDKNSQNIIADKLGTNRNDGNNLIILTAHLDSVNHSGGPSSNAPGADDNGSGSAGVLEIARIFKDVPTQHDLRFILFGGEEQGLLGSKQHVENLTTLDKNRIRAVINMDMIGTLNTSTPAVLIEGSPLSEEIINGLRNVASLYTNLDVKISLEPFDSDHVSFLNKEVPAVLTIEGEDSLNPNEHTANDTLEHIHYPLALDILRMNVAFIASQVTNLE

SEQ ID NO: 41 - protein, 471 aa, Bacillus thuringiensis
MGEEKMIKSLKQKMVSSLLAVSLAVGLAPIGQAKADSTSEITQTPSITKQIDANRAIEHIRFLSETIGPRPGGTKSEEWASRYIGMQLKSMGYEVEYQPFQVPDQYVGFIESPLSTKRNWQAGAAPNALISAEAVTAPLIFVKGGTKLADIPNEVNGKIVLFERGTTVADYNKQVENAVSKGAKGVLLYSLIGGRGNYGQTFNPRLMKKQSVPVFGLAYAQGNAFKEEIAKKGTTILSLKARHESNLTSLNVIAKKKPKNSTGNAKAVVISSHYDSVVGAPGANDNASGTGLVLELARALQNIETDKEIRFITFGSEETGLLGSDHYVNSLSQKERDRILGVFNADMVATNYDKAKNLYAMTPDGSTNLVTDAALQAGKQLNNNLVLQGKFGSSDHVPFAEVGIPAALFIWMGVDSWNPLIYHIEKVYHTPQDNVLENISPERMKMALDVIGTGVYNTLQTPVTQTEQKAA

SEQ ID NO: 42 - protein, 457 aa, Bacillus thuringiensis
MKKLTLIKKLIVTFGILLLFLFSNLSEDVILVHAMTKNDQSSLMSDKVRASRAIEHTRFLSEKIGPRPSGTKEEHKTATYIGDTLKKLGYEVEYQAFPVPDQYIGSISFSVYGEKEWQVGAASNASIPKDPVVGPVVSIGKENDIKNSPEPIHGKIVLLEQADTYKKYNEQVEYAAEKGAKAVLLYSTIGDRGNYGEAFNPVLKKKQSIPVFGLAYNQGIRMKDKIVHNKNIVSLKAKHEMNLSSLNVIAKKKPKYKTDNEDVAIMSAHYDSVIGAPGANDNASGVGLLLELAYNFKDIEINKEIQFIAFGAEENESLGAYHYVNQLSTTDKKHILGVFNADMIATSYQQAKDLYAMTLDGSQNEVTNTAVATSKALGKSVVLTGKFDSSDHVPFYEAGISSALFIWMDVESWNPLIYHVEKVFHTPQDTVLENISPQRMQEALEIIGTSLYYFIKR

SEQ ID NO: 43 - protein, 466 aa, Bacillus thuringiensis
MRKLLKQKIVSSLLAVSLAVSLAPIGQAKADSTSEVTQTPSITKQIDPNRAIEHVRFLSETIGPRPGGTKSEEWASRYIGMQLKSMGYDVEYQPFQVPDQYVGFIESPLSTKRNWQAGAAPNALISTEAVTAPLIFVQGGTKLEDIPNEVNGKIVLFERGTTVTDYNKQVENAVSKGAKGVLLYSLIGGRGNYGQTFNPHLMKKQSIPVFGLAYAQGNAFKEEIAKKGTTILSLKARHESKLTSLNVIAKKKPKKSTGNEKAVVVSSHYDSVVGAPGANDNASGTGLVLELARAFQNVETDKEIRFITFGSEETGLLGSDYYVNSLSQKERDRILGVFNADMVATNYDKAKNLYAMTSDGSTNFVTDAALQAGKQLNNNLILQGKFGSSDHVPFAEAGIPAALFIWMGVDSWNPLIYHIEKVYHTPQDNVLENISPERMRMALDVIGTGVYNSLQKPVPQTEQKAA

SEQ ID NO: 44 - protein, 445 aa, Brevibacillus sp.
MRSVRHYTLCALLVCGTILPIPFLSNRAHAEETQDWIDSERLYSHVEQLARTARPPATETEFAAAVYVENTLRSYGYKTKLDPFSYYTYRKPTTLSLTIEEWQGQKWTVSGFAFGINGTATAEVVDAGLGRAVDFANEASRGKIALVKRGEIPFGEKVRQAAAAGAVALIIWNDREGGWNASLGEPLDIAVPVIALSKADGTMLLKRIMDKRPVKGTIKVEGAATTKQTSYNIVASRKPNQNDTGQVVMVSAHHDSAGLSVGANHNGSGVAALLEIARSMADKPIDTEVRFVSFGAVTSGSRGPIAYANALSAKERQAMIAAFYVEGVGSQKAELIATNSLGNENLPIQLLRAAGVSPSNKDGTRERNGAAGYLAAAGIPTALVTSADTGQATDDSLVQMDWEQIATATKAVLSAIHKITEQTTPAYPMGSPNGGQLRAESEALQ

SEQ ID NO: 45 - protein, 446 aa, Brevibacillus sp.
MRSAGHYALGALLVWGSLLPLSFALPQAYANQARTGMDADLLYSHVERLAKTARPAATETEFAAGVYVENALRSYGYETALQPFYYHTYQKPASLSLAIEGWERSWAALPLTFGQNGAQTAELVDAGYGRAADFASGAARGKIALVKRGEIPFGEKVRQAAAAGAVAVIVWNDREGEWRASLGEPLDMSVPVIGLSREEGQLLRQRALKQPLRATVKVEGGVTRRHTAYNIIATKKPTEAATGQRVLILAHHDSAASSPGANDNASGVAVMLEVARKLSESQLDTEVQLVSFGAVTAGQRGQRAFSESLTEQDRQKIVAAYYVDAVGRSGDVTAFAGRREAVLATGLLAEAGAQQSALEVARAEQQRELAPLTVAGIPAVLVTRQKAAGEADRAGDVPATIEKEQLAEAAQMIFSAVGNITDTATPAYPIRSGQTTGQGEPEEDFQ

SEQ ID NO: 46 - protein, 297 aa, Flavobacterium sp.
MSKTFYKHDKYLSSDKLEGRFVGTKGNNNAAAYIKKYFKKYGLRQFNDSYYQPFKVFVKEGINKMKSDSVVTQNVIGYIEGSDAALKNEYIVIGAHYDHWGWGGQGSGSKKKEAFAIHNGADDNASGVSALLCILEEISKQKVKPKRSIIFISFSGEEEGLLGSKYFVNHLPVPKEAVKVMLNMDMVGRLNDKKELYMGGAGTFPNGVELMKKLQENSGLNPVIHAGDVGGSDHVSFYKESISAVGFHTGGHPQYHTPDDDIELINSDGGALVSKYIYNALIQIANYEEALVFIKQN

SEQ ID NO: 47 - protein, 411 aa, Streptomyces sp.
MYGFVAEALAAGGAPPAALLGPALGGVYLAVPAGARVEEVHRPGARPGDTECLLPDPALLTRPGEGAGGPRPGVRGAASGDGLPSAEVIAAVRATVTAEVLRGHVARLSGAEPLVEGEPLTVRSRDAAAEDNALVVEALAGRFQELGLRVRRHPFRWRGRQLFNVEAEHRVAGADSAVLVTAHLDSTASGGEFTDEDGAPRPYDPAVDPAPGADDDGSGTAGVLAAAECLASLLAAGRRPARSVRFVLFDAEEQGLVGSKFYARAAAAEGDRIAGVFQMDMIAGLRRGAVPRVEIHAGSAVPGPVVGASDALGELVARTLAAFDPAVTVQHLTGPGDPAVGRSDHASFHERGWAAVAVSEDLFSTPEGAPGTGTRQYHTPGDTLLDEDHDCGFAATIAGSVTATALAFAGL

SEQ ID NO: 48 - DNA, 975 bp, synthetic construct
  source 1..975: mol_type = other DNA
  misc_feature 1..975: note = Bacillus licheniformis truncated M28 family peptidase nucleotide sequence
atgaagagaaaaatgatgatgttcggattggcgctatcgatcattgcaggcggcgtggtcgccgatggaacggggaatgcagctcaagcggcccctcaggaaacagccatcgcaaaagatatcgaaaaattcagcaaaaaattcaacgagaaccgcgcctatcaaacgatttaccatttaagcgaaacgatcgggccgcgtgtgacaggcacggcagaagaaaaaaagagcgccgcttttatcgcctcacagatgaaaaagtcaaatctgaaagtaagcacacaaaagttcagcatacctgctcataaaaatcaaacctcgcaaaacgtaatcggcgtccgcaaagcaaaaggtgtcaaaaatcctgacatcgtttatgtgacttcgcattatgacagcgttccatatgcaccgggagccaatgacaatgcctccggcacttcagtcgtccttgaactggcccgaattttgaagacagtgccagccgacaaagaaattcgctttattacattcggagccgaagaaatcggactcctcggttcgcgccattatgtcagcacattgtcaaatcaggaagtcaaacggagcgttgccaactttaacttagatatggtggcgacaagctgggagaatgcttcacagctgtacatcaatacgcccgacggttcggcaaacctcgtctggcagctaagtaaagccgcttctttaagccttggaaaagacgtattatttttacatcaaggcggatcatccgaccatgtcccattccatgaagccggcatcgactcggccaactttatttggagagagccgggaacaggcgctttggagccttggtaccacaccccttacgacacgattgaacatatcagcaaagacaggytgaaaacagccggtcaaatcgcgggaacagctgtgtataacttcaccaagaaggaaaacagaaaaccgtcttacagctcagttgcccagtaa

SEQ ID NO: 49 - protein, 324 aa, synthetic construct
  REGION 1..324: note = Bacillus licheniformis truncated M28 family peptidase amino acid sequence
MKRKMMMFGLALSIIAGGVVADGTGNAAQAAPQETAIAKDIEKFSKKFNENRAYQTIYHLSETIGPRVTGTAEEKKSAAFIASQMKKSNLKVSTQKFSIPAHKNQTSQNVIGVRKAKGVKNPDIVYVTSHYDSVPYAPGANDNASGTSVVLELARILKTVPADKEIRFITFGAEEIGLLGSRHYVSTLSNQEVKRSVANFNLDMVATSWENASQLYINTPDGSANLVWQLSKAASLSLGKDVLFLHQGGSSDHVPFHEAGIDSANFIWREPGTGALEPWYHTPYDTIEHISKDRLKTAGQIAGTAVYNFTKKENRKPSYSSVAQ

SEQ ID NO: 50 - DNA, 74 bp, synthetic construct
  source 1..74: mol_type = other DNA
  misc_feature 1..74: note = oPlastBug-361 oligonucleotide
gtcaaatctgaaagtaagcacacaaaagttcagcatacctgctcataaaaatcaaacctcgcaaaacgtaatcg

SEQ ID NO: 51 - DNA, 72 bp, synthetic construct
  source 1..72: mol_type = other DNA
  misc_feature 1..72: note = oPlastBug-362 oligonucleotide
ggacgccgattacgttttgcgaggtttgatttttatgagcaggtatgctgaacttttgtgtgcttactttca

SEQ ID NO: 52 - protein, 327 aa, synthetic construct
  REGION 1..327: note = Bacillus licheniformis mature M28 family peptidase amino acid sequence with Yar-rowia lipolytica LIP2 signal peptide
MKLSTILFTACATIAAALPSPITPSEAAVLQKRAPQETAIAKDIEKFSKKFNENRAYQTIYHLSETIGPRVTGTAEEKKSAAFIASQMKKSNLKVSTQKFSIPAHKNQTSQNVIGVRKAKGVKNPDIVYVTSHYDSVPYAPGANDNASGTSVVLELARILKTVPADKEIRFITFGAEEIGLLGSRHYVSTLSNQEVKRSVANFNLDMVATSWENASQLYINTPDGSANLVWQLSKAASLSLGKDVLFLHQGGSSDHVPFHEAGIDSANFIWREPGTGALEPWYHTPYDTIEHISKDRLKTAGQIAGTAVYNFTKKENRKPSYSSVAQ

SEQ ID NO: 53 - DNA, 1359 bp, synthetic construct
  source 1..1359: mol_type = other DNA
  misc_feature 1..1359: note = Nucleotide sequence of Bacillus licheniformis mature M28 family peptidase amino acid sequence with Yarrowia lipolytica LIP2 signal peptide optimised to Yarrowia lipolytica
atgaagctgtctaccatcctgtttaccgcttgcgccaccatcgctgctgccctgccttcccccatcactccttctgaggccgctgtgctgcagaagcgagctccccaggagaccgctattgccaaggacatcgagaagttctccaagaagttcaacgagaaccgagcttaccagaccatctaccacctgtctgagaccattggacctcgagtgaccggaactgctgaggagaagaagtctgccgctttcatcgcctctcagatgaagaagtctaacctgaaggtctcgacccagaagttctcgatccctgatcgactggagggaaccctgactgtccagggcaacaacgttcccgctcgacctgccgctggatctgcccccactgctgccgagggcctggctgcccctctgtacgatgctggcctgggactgcctggagattttaccgaggaagctaagggaaagattgccgtgatcctgcgaggagagctgaccttctacgagaaggccaagaacgctgccgacgctggagcctctggcgtcattatctacaacaacgtcgactctctggtccccctcactcccaacctctctggaaacaaggtggacatccctgtcgttggagtcaagaaggaagacggagagaagctcctgtctgagcaggaagctattctgaagctgaaggctcacaagaaccagacctctcagaacgtgatcggagtgcgaaaggctaagggagtcaagaaccccgacatcgtgtacgtcacctctcactacgattctgttccctacgctcctggagccaacgacaacgcctctggaacttcggtggtcctggagctcgctcgaatcctgaagaccgtgcctgctgacaaggagatccgattcatcacctttggagccgaggagattggactcctgggctctcgacactacgtgtccactctctctaaccaggaagtcaagcgatctgtcgctaacttcaacctggatatggttgccacttcttgggagaacgcttctcagctctacattaacactcctgacggttctgctaacctcgtctggcagctgtctaaggccgcttctctgtctctcggaaaggacgtgctcttcctgcaccagggcggatcgtctgaccatgtgcctttccatgaggctggtattgactccgccaactttatctggcgagagcccggcactggtgccctcgagccttggtaccacactccttacgacaccatcgagcacatctccaaggatcgactcaagaccgctggccagattgccggcactgctgtctacaacttcaccaagaaggagaaccgaaagccttcctactcgtctgtcgcccagtga

SEQ ID NO: 54 - protein, 426 aa, synthetic construct
  REGION 1..426: note = : truncated Bacillus licheniformis mature M28 family peptidase amino acid se-quence with Yarrowia lipolytica LIP2 signal peptide
MKLSTILFTACATIAAALPSPITPSEAAVLQKRAPQETAIAKDIEKFSKKFNENRAYQTIYHLSETIGPRVTGTAEEKKSAAFIASQMKKSNLKVSTQKFSIPDRLEGTLTVQGNNVPARPAAGSAPTAAEGLAAPLYDAGLGLPGDFTEEAKGKIAVILRGELTFYEKAKNAADAGASGVIIYNNVDSLVPLTPNLSGNKVDIPVVGVKKEDGEKLLSEQEAILKLKAHKNQTSQNVIGVRKAKGVKNPDIVYVTSHYDSVPYAPGANDNASGTSVVLELARILKTVPADKEIRFITFGAEEIGLLGSRHYVSTLSNQEVKRSVANFNLDMVATSWENASQLYINTPDGSANLVWQLSKAASLSLGKDVLFLHQGGSSDHVPFHEAGIDSANFIWREPGTGALEPWYHTPYDTIEHISKDRLKTA

SEQ ID NO: 55 - DNA, 984 bp, synthetic construct
  source 1..984: mol_type = other DNA
  misc_feature 1..984: note = Nucleotide sequence of truncated Bacillus licheniformis mature M28 family pepti-dase amino acid sequence with Yarrowia lipolytica LIP2 signal peptide optimised to Yarrowia lipolytica
atgaagctgtctaccatcctgtttaccgcttgcgccaccatcgctgctgccctgccttcccccatcactccttctgaggccgctgtgctgcagaagcgagctccccaggagaccgctattgccaaggacatcgagaagttctccaagaagttcaacgagaaccgagcttaccagaccatctaccacctgtctgagaccattggacctcgagtgaccggaactgctgaggagaagaagtctgccgctttcatcgcctctcagatgaagaagtctaacctgaaggtctcgacccagaagttctcgatccctgctcacaagaaccagacctctcagaacgtgatcggagtgcgaaaggctaagggagtcaagaaccccgacatcgtgtacgtcacctctcactacgattctgttccctacgctcctggagccaacgacaacgcctctggaacttcggtggtcctggagctcgctcgaatcctgaagaccgtgcctgctgacaaggagatccgattcatcacctttggagccgaggagattggactcctgggctctcgacactacgtgtccactctctctaaccaggaagtcaagcgatctgtcgctaacttcaacctggatatggttgccacttcttgggagaacgcttctcagctctacattaacactcctgacggttctgctaacctcgtctggcagctgtctaaggccgcttctctgtctctcggaaaggacgtgctcttcctgcaccagggcggatcgtctgaccatgtgcctttccatgaggctggtattgactccgccaactttatctggcgagagcccggcactggtgccctcgagccttggtaccacactccttacgacaccatcgagcacatctccaaggatcgactcaagaccgctggccagattgccggcactgctgtctacaacttcaccaagaaggagaaccgaaagccttcctactcgtctgtcgcccagtga

SEQ ID NO: 56 - DNA, 67 bp, synthetic construct
  source 1..67: mol_type = other DNA
  misc_feature 1..67: note = oPlastBug-363 oligonucleotide
gtctaacctgaaggtctcgacccagaagttctcgatccctgctcacaagaaccagacctctcagaac

SEQ ID NO: 57 - DNA, 65 bp, synthetic construct
  source 1..65: mol_type = other DNA
  misc_feature 1..65: note = oPlastBug-364 oligonucleotide
gcactccgatcacgttctgagaggtctggttcttgtgagcagggatcgagaacttctgggtcgag